(19) United States
(12) Patent Application Publication
(10) Pub. No.: US 2018/0200487 A1
(43) Pub. Date: Jul. 19, 2018

(54) NEEDLE ASSEMBLIES WITH SAFETY SYSTEMS AND MANUFACTURING METHOD THEREFOR

(71) Applicant: B. Braun Melsungen AG, Melsungen (DE)

(72) Inventors: Stanislav Sokolski, Greifswald (DE); Hui Kuun Teoh, Penang (MY)

(21) Appl. No.: 15/742,006

(22) PCT Filed: Jul. 19, 2016
(86) PCT No.: PCT/EP2016/067193

§ 371 Date: Jan. 4, 2018

Related U.S. Application Data
(63) Continuation-in-part of application No. 14/803,984, filed on Jul. 20, 2015.

(51) Int. Cl. A61M 25/06; A61M 5/32

(52) U.S. Cl. CPC A61M 25/0618; A61M 5/326; A61M 2207/10; A61M 2005/3256; A61M 5/3275

(57) ABSTRACT

Safety needle assemblies that include a needle having a sharp distal tip and a needle tip cover mounted on the needle are disclosed. The needle tip cover includes a proximal wall having an opening for the needle to pass therethrough. The needle tip cover urges against the needle in a ready to use position and shields the sharp distal tip in the secured position. The needle tip cover is moved proximally by a biasing member after the needle has been inserted into a patient a predetermined depth.

### CROSS-REFERENCE TO RELATED APPLICATIONS

This is a National Phase application of PCT application No. PCT/EP2016/067193, filed Jul. 19, 2016, which claims priority to U.S. application Ser. No. 14/803,984, filed Jul. 20, 2015, the contents of each of which are expressly incorporated herein by reference.

### FIELD OF ART

The present invention is generally directed to needle safety assemblies and related methods and more particularly to needle assemblies with safety systems and related methods.

### BACKGROUND

Insertion procedures for any needle assembly typically includes the following basic steps: (1) the healthcare worker inserts the needle into the patient's vein or tissue; (2) after insertion into the vein with the needle tip, fluid is injected into the patient or drawn from the patient by pushing or pulling a plunger attached to the needle assembly; (3) the healthcare worker withdraws the needle by grasping the needle adapter end (opposite the pointed needle tip) while at the same time applying pressure to the patient's skin at the insertion site with a cotton swab in his or her free hand to stop the flow of blood; and (4) the healthcare worker then tapes the cotton swab to the patient's skin to stop the flow of blood and cover the puncture.

One potential problem with this procedure is that immediately after the withdrawal of the needle from the patient's vein, the healthcare worker, who is at this time involved in at least two urgent procedures, must place the exposed needle tip at a nearby location and address the tasks required to accomplish the needle withdrawal. It is at this juncture that the exposed needle tip creates a danger of an accidental needle stick, which, under the circumstances, can leave the healthcare worker vulnerable to the transmission of various dangerous blood-borne pathogens, such as HIV and hepatitis.

Other needle types similarly expose healthcare workers to risks of accidental needle sticks. For example, a doctor administering an injection using a straight needle, a Huber needle, an epidural needle, etc., may place the used needle on a tray for subsequent disposal by a nurse. For the period between placing the used needle on a tray or a work station to the time it is discarded, the used needle is a potential source for disease transmissions for those that work near or around the needle.

### SUMMARY

The various embodiments of needle assemblies have several features, no single one of which is solely responsible for their desirable attributes. Without limiting the scope of the present embodiments as set forth in the claims that follow, their more prominent features now will be discussed briefly.

A safety needle attachment, device, or assembly provided in accordance with aspects of the present disclosure can include a needle adapter, a needle, a needle guard or spring clip slidably carried or mounted on the needle, a biasing member positioned between the needle adapter and the spring clip, and a securing device compressing a portion of the biasing member in a ready to use position.

The biasing member can be a spring, a spring with coils, or a sleeve with biasing or resilient features, such as an elastic sleeve capable of elastic deformation and generating biasing forces.

The spring can be an extension spring that expands when compressed and then released. The sleeve can be an elongated elastomeric structure having resilient biasing properties. The sleeve can have a constant exterior wall or can be provided with joined arcuate sections. The spring and the sleeve can generically be referred to as a biasing member.

The needle adapter can have a proximal end and a distal end and may alternatively be referred to as a hub or a needle hub. The needle can project distally from the distal end of the needle adapter. The proximal end can have an opening for receiving a male medical implement, such as a syringe, for injecting fluid into the tissue of the patient or drawing fluid or blood from the patient.

The opening of the adapter can be a female Luer for receiving a male Luer tip, such as a syringe tip.

The exterior of the needle adapter can have exterior threads for threaded engagement with a threaded collar on the syringe or other medical implements.

A ledge can be provided at the interface of the needle and the distal end of the needle adapter.

An outer surface of the needle adapter can taper inward from the proximal end to the distal end to form a frustoconical shaped grip for a user to handily attach the safety needle assembly to a syringe or other medical devices.

The distal end of the frustoconical shaped grip can have a ring shaped planar surface defining the ledge.

The size and shape of the ledge may vary.

If no ledge is formed, the distal end of the needle adapter can be approximately the size of the needle shaft.

The size and the shape of the needle adapter can vary.

A cross-section of the needle adapter can be circular, rectangular, or any regular or irregular shape to form the grip. Surface features, such as fins and bumps, may be added to form a more effective gripping surface.

The needle can comprise a needle shaft having a proximal end attached to the needle adapter, a needle tip formed at a distal end of the needle shaft, and a change in profile positioned adjacent the needle tip on the needle shaft.

The proximal end of the needle shaft can be press fitted into the distal end of the needle adapter or glued into a glue well at the distal end of the needle adapter.

The needle tip can be configured to puncture the skin and tissue of a patient so that fluid can be injected into the patient, such as from a syringe attached to the needle adapter, or blood or other bodily fluid can be withdrawn through the needle tip into the syringe.

The needle tip can be cut or ground to have any known prior art tip geometries, including a non-coring needle tip.

The change in profile can have a cross-section larger than a cross-section of the nominal diameter of the needle shaft to prevent the spring clip from detaching from the safety needle assembly.

The change in profile can be a crimp, a bulge, a sleeve positioned around the needle shaft, an attachment, or a material buildup.

A spring can be provided with the needle to move a spring clip, needle guard, or tip protector in a distal direction to protect the tip from unintended needle sticks following use. The spring can allow the needle device to act as a passive safety device, not requiring additional steps beyond normal usage but still have unintended needle stick protection.

The spring can be a compression spring having a plurality of interconnected coils between the proximal end and the distal end of the spring. The spring can be a compression helical spring.

The proximal end of the spring can be placed in abutting contact with a ledge of the needle adapter or hub.

The proximal end most coil of the spring can be placed in abutting contact with the generally planar surface of the ledge.

To prevent the spring from separating from the needle adapter, the spring can be secured to the needle adapter, such as by mechanical inter-engagement, glue, bonding, or other securement means.

The proximal end of the spring can be attached directly to the needle shaft at or near the interface of the needle and the needle adapter.

The distal end of the spring can be attached to, pressed against, or in contact with the spring clip, or more specifically, to a proximally facing surface of a proximal wall of the spring clip to propel the spring clip distally towards the needle tip to shield the needle tip from inadvertent needle sticks.

The distal end of the spring can press against or abut the spring clip without mechanical inter-engagement so that the spring clip can remain rotatable about the needle shaft relative to the spring.

The distal end of the spring can be attached to the proximally facing surface of the proximal wall of the spring clip so that the spring clip cannot freely rotate about the needle shaft relative to the spring.

The spring can extend along nearly the entire length of the needle shaft from the ledge of the needle adapter towards the needle tip. In some examples, the length of the uncompressed spring is about 75-100% of the length of the needle shaft measured between the ledge and the needle tip. In a particular example, the length of the needle guard plus the length of the uncompressed spring is 101% or more of the length of the needle shaft measured between the ledge and the needle tip, such as 105% to about 125% of the length of the needle shaft measured between the ledge and the needle tip.

Characteristics of the spring, such as the spring material, coil diameter, wire diameter or thickness, and pitch can be selected depending on the particular application of the safety needle assembly.

A minimum spring constant may be achieved if the spring can extend nearly an entire length of the exposed needle shaft when the needle tip is secured by the spring clip.

The spring can have a latch point located between the proximal end and the distal end of the spring length. The latch point can be a portion or a point of a coil, a tab, a hook, a protrusion, or an attachment extending outwardly from a coil of the spring for use by a securing device to engage or latch onto.

When the securing device engages the latch point of the spring, the spring can be divided between an uncompressed portion distal of the latch point and a compressed portion proximal of the latch point.

The securing device can engage two latch points on the spring along two spaced positions on one coil or on two different coils. In other examples, one latch point or three or more latch points can be provided between the spring and the securing device.

The uncompressed portion of the spring can be defined between the latch point and the distal end of the spring, which can be contacted or abutted by a proximal wall of the spring clip.

The compressed portion of the spring can be defined between the proximal end and the latch point.

The entire length of the spring can be under compression, at least 80% of the entire uncompressed length of the spring can be under compression, or at least 50% of the entire length of the spring can be under compression.

The securing device, which may also be referred to as a hold back wire or a spring retainer, can comprise a mount disposed between the proximal end of the spring and the ledge, an attach end configured to engage with a latch point, and a securing arm coupling the attach end to the mount.

The mount can be attached directly to the needle adapter at a side of the ledge so that the proximal end of the spring can directly contact the ledge. In other examples, the mount can have flared or modified portions to enable the spring to seat there-against or to abut therewith instead of abutting the ledge directly.

The mount can have an opening for the needle to extend therethrough. The mount can be pivotably connected to a coil of the spring at or near the proximal end of the spring.

A securing arm can be pivotably attached to a mount via a pivot point.

The mount may embody a coiled loop and be unitarily formed with the securing arm with a bend between the mount and the securing arm defining a pivot point or section.

The securing arm may be pivotably mounted directly to the needle adapter without the need for a mount, such as being anchored directly into the wall surface of the needle adapter. For example, the needle adapter can have a flared section or protrusion and the securing arm attached directly thereto.

The securing arm may normally extend at least partially radially outward from the needle so that when the securing arm is pivoted about the pivot point and the attach end engages the latch point, elastic potential energy can be stored in the securing arm and/or the pivot point. That is, in the normal state when the attach end is not engaged, the securing arm and the attach end at the free end of the securing arm can be pointed away from the spring and the needle.

When the attach end is engaged with the latch point in the initial position, the securing arm can be simultaneously under tension caused by compressing the compressed portion of the spring, and elastically deflected inwardly towards the spring and the needle caused by pivoting or bending the securing arm towards the spring to engage the attach end with the latch point.

Elastic potential energy can be stored in the securing arm and/or the pivot point from the elastic deflection.

Tension in the securing arm caused by the spring under compression can maintain the engagement between the attach end and the latch point and prevent the securing arm and the attach end from elastically returning outwardly away from the spring.

The attach end can comprise a hook, a lever, a lip, a notch, a shoulder, a spline, or combinations thereof.

The attach end can removably engage the latch point and upon compression of the spring following the engagement, the latch point can separate and securing arm deflects radially outwardly from the latch point. The removable engagement can be a simple overlap with friction, a positive engagement with part of the attach end entering part of the latch point, or combinations thereof.

The location of the latch point between the proximal and distal ends of the spring can determine how far the spring clip can travel in the proximal direction, such as when pressed against and pushed by the skin in the proximal direction during an injection, before the attach end decouples from the latch point.

In some examples, the attach end engages the latch point at or near the distal most end of the spring. An extension is then provided with a flange or extending tab that abuts the needle guard. In use, the needle guard pushes against the extension that then pushes the spring in the proximal direction to separate the attach end from the latch point.

The latch point can be located at a point near a distal end of the spring so that a relatively small displacement of the spring clip in the proximal direction can decouple the attach end from the spring.

If the latch point is located near a proximal end of the spring, then a greater displacement of the spring clip may be required before the attach end is decoupled from the spring. Depending on the penetration depth of the needle into the patient, the location of the latch point and attach end along the length of the spring may be selected by the required proximal movement of the spring clip.

The attach end can be decoupled from the latch point of the spring by movement of the latch point relative to the attach end until contact between the latch point and the attach end ceases, decouples, or is otherwise removed or severed.

The attach end can be moved relative to the latch point to decouple the attach end from the latch point.

A decoupler can be incorporated to decouple the latch point from the attach end. In an example, the decoupler can press against the attach end and exert at least a radially outwardly force or component forces to separate the contact with the latch point.

The decoupler can be an adjacent coil immediately distal of the latch point.

The decoupler can contact and deflect the attach end radially outwardly, can compress the coils of the spring to move the latch point in a proximal direction away from the attach end, or separate the latch point and the attach end from one another.

The decoupler may be an attachment or protrusion extending from the adjacent coil distal of the latch point.

The decoupler may be a spring clip exerting a downward force or component forces to separate the latch point and the attach end from one another.

The decoupler can operate by contacting and pressing the attach end outwardly from the latch point until the attach end is no longer engaged with the latch point.

The decoupler can press against the latch point and move the latch point proximally until the attach end no longer engages the latch point.

The attach end can be shaped like a hook with the free end of the hook engaging with the latch point.

Because the compressed portion of the spring can be held in a compressed state by the attach end of the securing device, the safety needle assembly can be in an equilibrium state in the ready to use position.

The decoupler can be moved proximally when the needle is inserted into the patient thereby moving the spring clip proximally towards the latch point.

As the spring clip advances proximally on the needle, the uncompressed portion can start to compress and the decoupler advance towards the attach end.

Further proximal movement may cause the decoupler to press against the attach end and force the attach end to decouple or disengage from the latch point.

Further proximal movement may cause the compressed portion of the spring to further compress, which moves the latch point proximally away from the attach end.

The securing arm can elastically spring outwardly away from the spring due to the stored elastic potential energy and remain out of the path of the spring.

Once the attach end is no longer engaged with the latch point, the spring clip can further move proximally depending on the depth of the needle insertion and the compressed spring is ready to expand upon removal of the needle from the patient to move the tip protector or spring clip over the needle tip.

The spring clip can be prevented from moving distally by the securing device compressing the compressed portion of the spring in the ready to use position.

The spring clip can be configured to slide along the needle shaft to shield the needle tip from unintended needle pricks following activation, such as following use and the attach end is separated from the latch point.

The spring clip can slide proximally from the initial or ready to use position to an in-use position where the spring clip is further spaced from the needle tip, and then distally to the after-use, closed, protective, or secured position where the spring clip is effectively located over the needle tip to shield the needle tip, such as after the needle is removed from the patient.

The spring clip can comprises a proximal wall and two resilient arms extending distally from the proximal wall.

The spring clip can have a proximal wall having a proximally facing surface and a distally facing surface.

The proximal wall can be generally circular, round or rectangular and can include a circular or non-circular aperture or perimeter defining an opening for the needle to extend therethrough.

The opening can be sized larger than the needle shaft diameter but smaller than a size of the change in profile to prevent the proximal wall from moving distally past the change in profile in the secured position.

The spring clip can embody a number of different guard devices or configurations for covering and securing the needle tip from inadvertent needle sticks after withdrawing the safety needle following use.

Each resilient arm can comprise a distal wall having a free end biased against diametrically opposite portions or sides of the needle shaft when not in the secured position.

The resilient arm can have an elbow extending from the wall and a tip at the free end of the arm.

The tip can be a curved lip or hook which allows the free ends to slide smoothly along the needle shaft.

The tip can each have a simple straight edge with optional low friction material added to improve sliding relative to the surface of the needle, such as medical grade lubricant.

The free ends of the spring clip can be slidably positioned on opposite sides of the needle shaft to allow the spring clip to slide axially while minimizing any moment about the needle.

The tips of the two resilient arms can press against the needle shaft in the ready to use position and the in-use position.

The resilient arms can be elastically deflected outwardly by the needle shaft and the ends of the distal walls can be pressed against diametrically opposite points of the needle shaft.

When the needle is inserted into the patient, the spring clip can move proximally along the needle shaft by abutting against the skin of the patient.

When the needle is inserted deeper into the patient, the spring clip can move proximally thereby compressing the uncompressed portion of the spring and bringing the decoupler closer to the latch point.

Eventually, the decoupler of the spring may press against the attach end or move the latch point proximally of the attach end to disengage the attach end from the latch point.

Once the attach end is no longer engaged with the latch point, the elastic securing arm can elastically spring outwardly away from the spring and remain displaced from the spring. At this point, the spring clip may be pressed against the skin with the full length of the spring unrestricted by the securing device.

As the needle is removed from the patient, the spring clip can slide distally along the needle shaft by the expansion force of the spring until the proximal wall or the perimeter defining the opening on the proximal wall contacts the change in profile on the needle to stop further distal movement of the spring clip and further expansion of the spring. Before, concurrently, or shortly thereafter, the resilient arms of the spring clip, may no longer press against the needle shaft, activate, and move radially to move the two distal walls over the needle tip to cover the needle tip in the secured position.

In the secured position, the free ends or the tips of the resilient arms can slide off the needle shaft and elastically move towards each other to cover the needle tip to prevent inadvertent needle sticks.

The distal walls can each have folded tabs, such as two or more folded tabs, to define a holding space for capturing the needle tip therein to prevent the needle tip from moving laterally outside the side edges of the resilient arms.

The resilient arms can have different lengths so that when the spring clip is activated, the two distal walls are axially offset and covering the needle tip.

The engagement between the change in profile and the opening in the proximal wall of the spring clip can prevent the spring clip from displacing distally off of the needle.

The distally facing surface, such as the perimeter defining the opening, of the proximal wall can abut against the change in profile to prevent further distal movement of the spring clip following movement to the used or protected position in which the needle tip is shielded.

Because the change in profile is larger in size than the opening, the spring clip may not slide past the change in profile, thereby confining the needle tip within the spring clip and preventing unintended needle sticks after the needle has been removed from the patient.

A cylinder or housing having a cavity can accommodate the spring clip.

The housing can resemble a cup having an elongated wall, one close end with an opening for the needle and one open end, which can be larger in size than the opening on the closed end.

The housing can be placed at a distal end of the spring or somewhere near the distal end.

The housing can be sized and shaped to accept the spring clip therein and allow the arm of the spring clip to move between the ready to use position and the protective position.

The housing can have a distal end edge that extends distal of the spring clip so that when the needle is used on a patient, the skin pushes against the distal end edge of the housing and not the spring clip.

The elongated wall can be a partially continuous, such as being semi-enclosed or having only part of a circumference.

The securing device can comprise two attach ends, each attach end having an engaging surface and a pressing surface angled from the engaging surface.

The securing arm can extend from the engaging surface of each attach end and can be pivotally attached to the mount or the needle adapter at the pivot point.

The mount can be sandwiched between the proximal end of the spring and the ledge of the needle adapter.

Each attach end can be engaged with a latch point located on opposite sides of the spring to secure the needle assembly in a ready to use position.

The spring can have a compressed portion between the latch points and the proximal end of the spring and an uncompressed portion between the latch point and the spring clip.

The position of the latch points for the two attach ends along the length of the spring can be closer to the distal end of the spring than the proximal end of the spring.

The latch points can be positioned anywhere along the length of the spring depending on the depth of needle insertion into the patient.

The position of the latch points relative to the length of the spring can be selectable depending on the desired needle application.

The latch points can be axially offset to match with axially offset attach ends.

Decouplers can be incorporated and located on an adjacent coil distal of the coil with the latch points.

The decouplers can be located on the same distal coil or different coils.

The latch points can be staggered along the length of the spring such that the securing arms have unequal lengths to form a first and second compressed portions and one uncompressed portion.

A first latch point can be located closer to the distal end of the spring and a second latch point can be located between the first latch point and the proximal end of the spring.

The first compressed portion can be defined between the first latch point and the second latch point, and the second compressed portion can be located between the second latch point and the proximal end of the spring.

In the first stage, the first decoupler can disengage the first attach end from the first latch point so that the first compressed portion and the uncompressed portion combine to form a new uncompressed portion.

In the second stage, the second decoupler can disengage the second attach end from the second latch point to fully release the spring from the securing mechanism.

The shape of each of the attach ends can also affect the required displacement of the decoupler, the spring clip, the housing for accommodating the spring clip, or all separation between the release ends and the attach points.

The shape and size of the attach end can determine how far the decoupler must travel before abutting and pushing the attach end and decoupling the attach end from the latch point of the spring.

If the pressing surface is closer to the decoupler of the spring or had a different angle in the ready to use position, then a smaller amount of movement of the spring clip may release the attached points to free the compressed portion of the spring than if the pressing surface is further proximally away from the decoupler.

The needle device can be practiced with a single attach end for attaching to a single latch point.

There can be three or more attached ends for attaching to three or more attach points.

When the needle is inserted into the patient, the spring clip can be moved proximally towards the two latch points.

As the spring clip moves proximally towards the latch points, the decouplers located on an adjacent coil distal of the latch points can strike the pressing surfaces of the attach ends.

Because the pressing surface may be angled with respect to the engaging surface, the proximal movement of the decoupler pushes the attach end outwardly away from the spring, due to component forces, until the engaging surface is no longer engaging the latch point. At this point, the two securing arms and the two attach end may elastically spring outwardly away from the spring and release the compressed portion of the spring.

Two decouplers can be axially offset or staggered so that one pair of securing arms and attach ends will release prior to the release of the second pair of securing arms and attach ends.

If the latch points are staggered or axially spaced, the first compressed portion on one side of the spring may be released when the first decoupler disengages the first attach end from the first latch point and the second compressed portion on the opposite side of the spring may be released when the second decoupler disengages the second attach end from the second latch point.

As the needle is withdrawn from the patient, the spring clip may be advanced distally by the spring towards the needle tip until the needle tip is covered by the spring clip.

Distal movement of the spring clip can be stopped by the proximal wall engaging the change in profile near the needle tip, as previously discussed with reference to the first embodiment.

The attach ends and the latch points can be located closer to the proximal end of the spring.

The attach ends can compress about half the length of the spring.

The attach ends can compress less than half the length of the spring.

The longer the uncompressed portion than the compressed portion, the longer the spring clip can be displaced before the decouplers disengage the attach ends from the latch points.

A longer uncompressed portion can allow the spring clip to travel proximally a longer distance before the attach end releases the compressed portion of the spring to allow the needle to penetrate deeper into the patient before the compressed portion of the spring is released.

When the needle is inserted into the patient, the spring clip can move proximally thereby compressing the uncompressed portion of the spring.

Because the uncompressed portion is longer than the compressed portion, the amount of force required to move the spring clip proximally may be less than if the uncompressed portion was shorter, assuming other spring characteristics being the same.

The longer the uncompressed portion, the less the pressure may be applied by the spring clip against the patient.

The spring constant of the spring and the choice of material of the spring may also affect the overall pressure of the spring clip against the patient when inserting the needle into the patient.

As the spring clip is moved proximally towards the needle adapter by pressing against the skin of the patient, the decoupler may push against the pressing surfaces of the two attach ends thereby forcing the attach ends down and away from the latch points of the spring until both attach ends are decoupled from the latch points of the spring.

As each attach end is decoupled from the part of the spring, the flexible arm may spring outwardly away from the spring.

As the needle is removed from the patient, the spring clip may move distally under spring force towards the needle tip while providing resistance against the patient with decreasing force.

When the needle is completely removed from the patient, the ends of the spring clip may move distal of the needle tip and elastically spring back towards each other to cover the needle tip.

The change in profile can prevent the spring clip from further distal movement to prevent removing the spring clip from the needle.

The safety needle assembly can comprises a needle adapter, a needle, a needle guard or spring clip slidably carried on the needle, an elastic sleeve extending distal the needle adapter and enclosing the spring clip and a portion of the needle proximal of the spring clip, and a securing device compressing at least a portion of the elastic sleeve in a ready to use position.

The needle adapter can have a proximal end and a distal end and may alternatively be referred to as a hub or needle hub.

The needle can project distally from the distal end of the needle adapter. The proximal end can have an opening for receiving a male medical implement such as a syringe for injecting fluid into the tissue of the patient or drawing fluid or blood from the patient.

The opening can be a female Luer for receiving a male Luer tip, such as a syringe tip.

The exterior of the needle adapter can have exterior threads for threaded engagement with a threaded collar on the syringe or other medical implements.

A ledge may or may not be formed adjacent the distal end of the needle adapter. The overall shape of the needle adapter may vary and therefore the size and shape of the ledge may also vary.

An outer surface of the needle adapter can have portions which taper inward from the proximal end to the distal end to form a partially cylindrical and partially frustoconical shaped grip for a user to handily attach the safety needle assembly to a syringe or other medical devices.

Adjacent the distal end of the frustoconical shaped grip can be a ring shaped planar surface defining the ledge for supporting the elastic sleeve.

A cross-section of the needle adapter can be circular, rectangular, or any regular or irregular shape to form the grip.

The shape of the needle adapter is not restricted and can vary depending on the needs and use of the safety needle assembly.

Surface features, such as fins and bumps, may be added to form a more effective gripping surface.

A catch can extend from the outer surface of the needle adapter between the proximal end and the distal end of the needle adapter.

The securing device can extend from an outer surface of the sleeve and engages the catch to compress a portion of the sleeve to expose the needle tip in the ready to use position.

The securing device can be provided on the outer surface of the needle adapter to engage a catch extending from the outer surface of the sleeve in the ready to use position.

The elastic sleeve can be provided with the needle to act as a passive safety device configured to move the spring clip or tip protector in a distal direction to protect the tip from unintended needle sticks following use.

The sleeve can have one axially fixed end and one movable end. When the movable end is moved, the sleeve is compressed or is further compressed from an initial compressed position. When compressed or when further compressed, the sleeve goes from a first length to a second length, which is smaller than the first length. The sleeve can be called or function as a biasing member or element.

The elastic sleeve can prevent or significantly reduce the likelihood of blood exposure when covering the needle tip.

The elastic sleeve can be a hollow tubular structure defining an interior cavity having an opening at a proximal end and a closed distal end with an aperture for the needle to extend therethrough in the ready to use position.

The sleeve can be sleeved over the needle and the spring clip with the needle tip extending through the aperture and the opening surrounding the distal end of the needle adapter.

To prevent the elastic sleeve from separating from the needle adapter, the proximal end of the elastic sleeve can be coupled to the needle adapter, such as at the ledge of the needle adapter by mechanical inter-engagement, glue, bonding, welding, ultrasonic welding, or other securement means.

The proximal end of the sleeve can be attached directly to the needle shaft at or near the interface of the needle and the needle adapter.

A necked portion can be adjacent the distal end of the sleeve and proximal of the spring clip. The necked portion may be a portion of the sleeve that extends inwardly towards the needle to form a reduced passage, which has a size or diameter less than that of the interior cavity of the sleeve to confine the spring clip in the interior cavity between the distal end of the sleeve and the necked portion. The necked portion can also be formed by providing a disc or a flange on the interior of the sleeve with a passage or an orifice for the passage of the needle.

In an example, the necked portion can be a gradual surface change inside the sleeve, a disc or flange forming a barrier to retain the spring clip or tip protector inside the sleeve.

The necked portion can be configured to press against the spring clip, or more specifically, to a proximally facing surface of a proximal wall of the spring clip to propel the spring clip distally towards the needle tip to shield the needle tip from inadvertent needle sticks.

The necked portion can be an internal shoulder extending radially inwardly from a surface of the interior cavity of the sleeve towards the needle thereby forming a passage large enough for the needle shaft to pass through and small enough to advance the spring clip along the needle shaft.

The size or diameter of the orifice can be slightly larger than the needle shaft and less than the proximal wall of the spring clip.

The necked portion of the sleeve can press against or abut the spring clip without mechanical inter-engagement so that the spring clip can rotate about the needle shaft relative to the sleeve.

The necked portion can be attached to the proximally facing surface of the proximal wall so that the spring clip is not free to rotate about the needle shaft relative to the sleeve.

The sleeve can extend along nearly the entire length of the needle shaft from the ledge of the needle adapter.

The sleeve can function as a spring surrounding both the spring clip and at least partially the needle.

The characteristics of the sleeve, such as the sleeve material and elastic properties of the sleeve, can be selected depending on the particular application of the safety needle assembly.

When a force is applied to the sleeve to compress the sleeve some distance, elastic energy is stored in the sleeve. When the force is removed, the sleeve can elastically return to its original shape.

The applied force required to compress the sleeve may be proportional to the distance compressed by a spring rate, which can be linear or non-linear. A linear spring rate may have a spring constant meaning the compression of the sleeve can be directly proportional to the applied force.

A sleeve having a high spring constant may require a larger applied force than a sleeve having a smaller spring constant to compress the sleeve.

The securing device, which may also be referred to as a hold back strap, can comprise an arm extending from the outer surface of the sleeve and an attach end at a free end of the arm.

The arm and the attach end can be integrally formed with the sleeve or attached to the sleeve by mechanical inter-engagement, glue, bonding, welding, or other securement means.

The arm of the securing device can extend proximally in its natural state from the sleeve.

The arm of the securing device can extend radially outwardly with respect to the needle axis in its natural state from the sleeve to ensure the attach end at the free end of the arm is moved away from the catch to prevent the attach end from reengaging the catch when the attach end disengages from the catch.

The arm can have a bore extending through a body of the arm between the attach end and the sleeve, and an elastic rod inserted inside the bore to ensure the arm extends at least partially radially outwardly in its natural state.

The arm can comprise an elastic sleeve fitted over the arm to extend the arm at least partially radially outwardly in its natural state.

The attach end engages the catch on the needle adapter when the safety device is in the ready to use position. The catch can have an anchor end extending from the needle adaptor.

When the sleeve is compressed a certain distance, the attach end can disengage from the catch and spring radially outwardly to its natural state from the release of the stored elastic energy in the arm.

The arm can extend outwardly from the outer surface of the sleeve adjacent to or at the distal end of the sleeve.

The arm can extend outwardly from the sleeve adjacent to the necked portion of the sleeve.

The location of the arm along the length of the sleeve can determine the amount of force applied to the sleeve and the amount of compression required before the attach end disengages from the catch.

The catch can be a tab, a hook, a protrusion, or an attachment extending outwardly from the needle adapter for the securing device to latch onto. The end of the catch connected to the needle adapter may be referred to as an anchor end. The anchor end can be unitarily formed with the needle adapter. In other examples, the anchor end is secured to the needle adapter, such as by welding, gluing, co-molding, or insert molding.

The catch can be located between the proximal end and the distal end of the needle adapter.

The catch can be located proximal the ledge.

The catch can be located on the sleeve proximal of the arm and the attach end.

When the attach end of the securing device engages the catch, the elastic sleeve may be at least partially compressed to provide tension along the securing device to maintain the attach end to the catch.

When the securing device engages the catch, the sleeve can be divided between an uncompressed portion distal of an anchor point, where the arm extends from the sleeve, and a compressed portion proximal of the anchor point.

The uncompressed portion of the sleeve can be defined between the anchor point and the distal end of the sleeve.

The uncompressed portion may be at least partially compressed when the needle is inserted into the patient.

The uncompressed portion can be incompressible so that the uncompressed portion would not squeeze the spring clip inside the uncompressed portion and possible damage or affect operation of the spring clip.

The anchor point can be located at or near the necked portion of the sleeve so that only the portion of the sleeve proximal of the anchor point is allowed to compress in the ready to use position.

The compressed portion of the spring can be defined between the proximal end of the sleeve at the ledge and the anchor point.

Nearly the entire length of the sleeve proximal of the spring clip can be under compression, at least 80% of the entire uncompressed length of the sleeve can be under compression, or at least 50% of the entire length of the sleeve can be under compression.

The securing device may normally extend at least partially radially outwardly with respect to the axis of the needle so that when the attach end of the securing device engages the catch, elastic potential energy can be stored in the securing device.

The arm and the attach end at the free end of the arm can be pointed away from the sleeve and the needle, so that when the attach end is engaged with the catch in the initial position, the arm can be simultaneously under tension caused by compressing the compressed portion of the sleeve and elastically deflected inwardly towards the sleeve and the needle caused by pivoting or bending the arm towards the sleeve to engage the attach end with the catch.

Elastic potential energy can be stored in the arm from the elastic deflection.

The tension in the arm can be caused by the sleeve under compression to maintain the engagement between the attach end and the catch and prevents the arm and the attach end from elastically returning outwardly away from the sleeve.

The sleeve can be maintained under compression in the ready to use position by the securing device, which can function as a hold back strap.

The attach end can comprise a loop, a hook, a lever, a lip, a notch, a shoulder, a spline, or combinations thereof.

The attach end can removably engage the catch and upon compression of the sleeve, separate and deflect radially outwardly from the catch. The removable engagement can be a simple overlap with friction, a positive engagement with part of the catch entering part of the attach end, or combinations thereof.

The catch can be a tab, a hook, a protrusion, a notch, or an attachment angled at least partially proximally so that the attach end can slip off easily when the sleeve is further compressed as the needle is inserted into the patient.

The securing device can be coupled to the catch via the attach end thereby dividing the sleeve into the compressed portion proximal of the anchor point and the uncompressed portion distal of the anchor point in the ready to use position.

The location of the anchor point between the proximal and distal ends of the sleeve can determine how far the sleeve and/or the spring clip in the sleeve can travel in the proximal direction, such as when the distal end of the sleeve is pressed against and pushed by the skin in the proximal direction during an injection, before the attach end decouples from the catch.

The anchor point can be located near a distal end of the sleeve adjacent to the necked portion, so a relatively small displacement of the sleeve and/or the spring clip in the proximal direction can decouple the attach end from the sleeve.

If the anchor point is located closer to a proximal end of the sleeve, then a greater displacement of the spring clip may be required before the attach end can be decoupled from the sleeve.

Depending on the penetration depth of the needle into the patient, the location of the anchor point along the length of the sleeve and the distance required to disengage the attach end from the catch can be selected by the required proximal movement of the spring clip.

The attach end can be decoupled from the catch by movement of the attach end relative to the catch until contact between the catch and the attach end is removed.

The attach end can be shaped like a closed loop with the catch extending through the loop.

The attach end can be shaped like a hook and the catch can be shaped like a closed loop or a ledge without or with a lip extending in a direction opposite the hook-shaped attach end to positively engage the attach end in the ready to use position.

Because the compressed portion of the sleeve is held in a compressed state by the engagement of the attach end with the catch, the safety needle assembly can be in an equilibrium state in the ready to use position.

The securing device can be moved proximally when the needle is inserted into the patient thereby moving the distal end of the sleeve and the spring clip proximally towards the catch.

As the distal end of the sleeve and the spring clip advance proximally on the needle, the uncompressed portion may start to compress, if compressible, and the attach end advances distally away from the catch thereby breaking contact with the catch and eventually out of the reach of the catch so that the arm can elastically spring outwardly away from the sleeve due to the stored elastic potential energy and thereafter remain out of the reach of the catch.

Once the attach end is no longer engaged with the catch, the sleeve and the spring clip inside the sleeve can further move proximally depending on the depth of the needle insertion.

The sleeve can be ready to expand upon removal of the needle from the patient to move the spring clip or tip protector or spring clip over the needle tip.

The spring clip can be prevented from moving distally by the sleeve in the ready to use position.

The spring clip can be configured to slide along the needle shaft to shield the needle tip from unintended needle pricks following activation, such as following use and the attach end is separated from the catch.

The spring clip can slide proximally from the initial or ready to use to an in-use position where the spring clip can be further spaced from the needle tip, and then distally to the after-use, closed, protective, or secured position where the spring clip is effectively located over the needle tip to shield the needle tip, such as after the needle is removed from the patient.

When the needle is inserted into the patient, the distal end of the housing, which is the distal end of the sleeve can push against the skin of the patient.

The spring clip can be moved proximally along the needle shaft by abutting against the inside surface of the interior cavity of the sleeve at the distal end of the sleeve.

As the needle is inserted deeper into the patient, the spring clip can be pushed along proximally along the needle shaft by the distal end of the sleeve while further compressing the compressed portion of the sleeve to move the attach end proximally and away from the catch.

The attach end can disengage from the catch as the needle extends deeper into the patient, at which time the arm and the attach end elastically spring outwardly away from the catch and remain displaced from the catch.

The distal end of the sleeve can be pressed against the skin with at least part of the sleeve under compression.

As the needle is removed from the patient, the spring clip can be urged distally along the needle shaft by the necked portion of the sleeve pressing against the proximal wall of the spring clip until the proximal wall, and more specifically the perimeter defining the opening on the proximal wall, contacts the change in profile on the needle, which stops further distal movement of the spring clip and further expansion of the sleeve.

Before, concurrently, or shortly thereafter, the resilient arms of the spring clip, now no longer pressing against the needle shaft, can activate and move radially to move the two distal walls over the needle tip to cover the needle tip in the secured position.

In the secured position, the free ends or the tips of the resilient arms can slide off the needle shaft and elastically move towards each other to cover the needle tip to prevent inadvertent needle sticks.

The distal walls can each have folded tabs, such as two or more folded tabs, to define a holding space for capturing the needle tip therein to prevent the needle tip from moving laterally outside the side edges of the resilient arms.

The resilient arms can also have different lengths so that when the spring clip is activated, the two distal walls are axially offset and covering the needle tip.

If no change in profile on the needle is provided, the distal end of the sleeve, when the sleeve is fully expanded to its natural state, can stop further distal movement of the spring clip when the spring clip is activated and covering the needle tip.

The engagement between the change in profile and the opening in the proximal wall of the spring clip or the distal end of the sleeve can prevent the spring clip from displacing distally off of the needle.

The distally facing surface, such as the perimeter defining the opening of the proximal wall can abut against the change in profile to prevent further distal movement of the spring clip following movement to the used or protected position in which the needle tip is shielded.

Because the change in profile can be larger in size than the opening, the spring clip cannot slide past the change in profile, thereby confining the needle tip within the spring clip and preventing unintended needle sticks after the needle has been removed from the patient.

Because the aperture at the distal end of the sleeve is smaller than the spring clip, the spring clip is confined within the interior cavity of the sleeve between the distal end the sleeve and the necked portion.

The distal portion of the sleeve between the distal end 484 of the sleeve and the necked portion can function as a housing having an interior cavity for accommodating the spring clip.

The housing can be sized and shaped to accept the spring clip therein and allow the resilient arms of the spring clip to move between the ready to use position and the protective position.

The safety needle assembly can comprise a pair of securing devices extending from opposite sides of the sleeve, and a pair of catches extending from opposite sides of the needle adapter engaging the pair of securing devices to hold back the elastic sleeve under compression in the ready to use position.

The pair of catches may function as release buttons, which when activated by a user, disengage the pair of catches from the pair of securing devices.

The pair of catches can be activated by squeezing each of the pair of catches towards each other simultaneously.

The safety needle assembly can be an active device that requires the user to release the sleeve and the spring clip inside the sleeve to cover the needle tip in the protective position upon needle removal by activating the catches removably coupled to the securing devices.

The safety needle assembly can comprise a needle adapter, a needle, a needle guard or spring clip slidably carried on the needle, the elastic sleeve extending distal the needle adapter and enclosing the spring clip and a portion of the needle proximal of the spring clip, and the pair of securing devices compressing at least a portion of the elastic sleeve in a ready to use position.

The entire length of the elastic sleeve distal the needle adapter can be compressed.

The pair of catches can each extend from opposite ends of the outer surface of the needle adapter between the proximal end and the distal end.

The overall shape of the needle adapter may vary.

At least some portions of an outer surface of the needle adapter can taper inward from the proximal end to the distal end to form at least a partially frustoconical shaped grip for a user to handily attach the safety needle assembly to a syringe or other medical devices.

A cross-section of the needle adapter 420 can be circular, rectangular, or any regular or irregular shape to form the grip.

Surface features, such as fins and bumps, may be added to form a more effective gripping surface.

The proximal end of the sleeve can be attached around a distal portion of the outer surface of the needle adapter.

Adjacent the distal end of the needle adapter can be a ring shaped planar surface defining the ledge for supporting the elastic sleeve.

The shape and size of the needle adapter is not limited.

The needle can comprise a needle shaft having a proximal end attached to the needle adapter, a needle tip formed at a distal end of the needle shaft, and, optionally, a change in profile positioned adjacent the needle tip on the needle shaft.

The elastic sleeve can be provided with the needle to act as a passive safety device configured to move the spring clip or tip protector in a distal direction to protect the tip from unintended needle sticks following use.

The elastic sleeve can be a hollow tubular structure defining an interior cavity having an opening at a proximal end and a closed distal end with an aperture for the needle to extend therethrough in the ready to use position.

The sleeve can be sleeved over the needle and the spring clip with the needle tip extending through the aperture and the opening surrounding the distal end of the needle adapter.

To prevent the elastic sleeve from separating from the needle adapter, the proximal end of the elastic sleeve can be secured to the ledge of the needle adapter such as by mechanical inter-engagement, glue, bonding, welding, ultrasonic welding, or other securement means.

The proximal end of the sleeve can be attached directly to the needle shaft at or near the interface of the needle and the needle adapter.

A necked portion adjacent the distal end of the sleeve and proximal of the spring clip can be configured to press against the spring clip, or more specifically, to a proximally facing surface of a proximal wall of the spring clip to propel the spring clip distally towards the needle tip to shield the needle tip from inadvertent needle sticks.

The necked portion can be an internal shoulder, which can extend radially inwardly from a surface of the interior cavity of the sleeve towards the needle thereby forming a passage large enough for the needle shaft to pass through and small enough to advance the spring clip along the needle shaft.

The size or diameter of the orifice can be slightly larger than the needle shaft and less than the proximal wall of the spring clip.

The necked portion of the sleeve can press against or abut the spring clip without mechanical engagement so that the spring clip can remain freely rotatable about the needle shaft relative to the sleeve.

The necked portion can be attached to the proximally facing surface of the proximal wall so that the spring clip is not free to rotate about the needle shaft relative to the sleeve.

The sleeve can extend along nearly the entire length of the needle shaft from the ledge of the needle adapter.

Characteristics of the sleeve, such as the sleeve material and elastic properties of the sleeve, can be selected depending on the particular application of the safety needle assembly.

When a force is applied to the sleeve to compress the sleeve some distance, elastic energy can be stored in the sleeve. When the force is removed, the sleeve can elastically return to its original shape.

The securing devices, which can be referred to as hold back straps, can each comprise an arm extending from the outer surface of the sleeve and an attach end at a free end of the arm.

The arm and the attach end can be integrally formed with the sleeve or attached to the sleeve by mechanical inter-engagement, glue, bonding, welding, or other securement means.

The arm of the securing device can extend proximally and may or may not extend radially outwardly with respect to the needle axis in its natural state from the sleeve.

The arm may coil up in its natural state thereby shortening the overall length of the arm when the securing arm disengages from a corresponding catch.

The arm can extend outwardly from the outer surface of the sleeve adjacent to or at the distal end of the sleeve.

The arm can extend outwardly from the sleeve adjacent to the necked portion of the sleeve to prevent compression of the sleeve surrounding the spring clip.

The catch can be an elastic protrusion extending distal the proximal end of the needle adapter between the proximal end and the distal end of the needle adapter.

The catch can also extend at least partially radially outwardly in its natural state so that when the catch is deflected inwardly towards the needle axis in an engaging position to hold the attach end of the securing device in the ready to use position, elastic potential energy can be stored in the catch.

A free end of the catch can directly contact a holding device located on the needle adapter or elastic sleeve to maintain the catch in the engaging position.

The holding device can be a notch on the needle adapter or sleeve, or the corner formed between the proximal end of the sleeve and the needle adapter.

The catch can spring from the engaging position to an activated position, which can be a position of the catch in its natural state, by removing contact between the free end of the catch and the holding device, such as by squeezing the catches towards each other.

The catch may also be a clamp or other holding device on the needle adapter capable of maintaining the sleeve under compression in the ready to use position by securing the attach end of the securing device and releasing the attach end of the securing device to allow the sleeve to expand to its normal state.

When the attach end of the securing device engages the catch, the elastic sleeve is at least partially compressed to provide tension along the securing device to maintain the attach end to the catch.

When the securing device engages the catch, the sleeve can be divided between an uncompressed portion distal of an anchor point, where the arm extends from the sleeve, and a compressed portion proximal of the anchor point.

The uncompressed portion of the sleeve can be defined between the anchor point and the distal end of the sleeve.

The anchor point can be located at the distal end of the sleeve, in which case there is no uncompressed portion.

The anchor point may be near the necked portion of the sleeve so that the portion of the sleeve proximal of the anchor point is allowed to compress.

The compressed portion of the spring can be defined between the proximal end of the sleeve and the anchor point.

Nearly the entire length of the sleeve proximal of the spring clip can be under compression, at least 80% of the entire uncompressed length of the sleeve can be under compression, or at least 50% of the entire length of the sleeve can be under compression.

The arm of the securing device may normally be straight or coiled so that when the attach end of the securing device engages the catch, elastic potential energy is stored in the arm of the securing device.

When the arm is engaged with the catch in the initial position, the arm is simultaneously under tension caused by compressing the compressed portion of the sleeve thereby elastically stretching out the arm into a straight span if normally coiled.

Elastic potential energy can be stored in the arm from the elastic deflection.

The tension in the arm caused by the sleeve under compression can maintain the engagement between the attach end and the catch and prevent the arm and the attach end from elastically returning outwardly away from the sleeve.

The sleeve can be maintained under compression in the ready to use position by the securing device, which functions as a hold back strap.

The attach end can comprise a loop, a hook, a lever, a lip, a notch, a shoulder, a spline, or combinations thereof.

The attach end can removably engage the catch and upon compression of the sleeve, separate and pull away from the catch by elongation of the sleeve as it returns to its normal state.

The removable engagement can be a simple overlap with friction, a positive engagement with part of the catch entering part of the attach end, or combinations thereof.

Because the compressed portion of the sleeve is held in a compressed state by the engagement of the holding device with the catch, the safety needle assembly can be in an equilibrium state in the ready to use position.

When the catches are activated, the catches can break contact with the holding device thereby releasing the attach end of the securing device.

Depending on the structure of the catch, the catch can elastically spring outwardly away from the securing device due to the stored elastic potential energy and thereafter remain out of the reach of the securing device.

Once the attach end is no longer held by the catch, the sleeve can be restored to its natural state and push the spring clip inside the sleeve to move distally to cover the needle tip.

The sleeve can confine the spring clip and prevent the spring clip from moving distally in the ready to use position.

The spring clip can be configured to slide along the needle shaft to shield the needle tip from unintended needle pricks following activation, such as following use and the attach end is separated from the catch.

The spring clip can slide proximally from the initial or ready to use position to the after-use, closed, protective, or secured position where the spring clip is effectively located over the needle tip to shield the needle tip, such as after the needle is removed from the patient.

As the needle is removed from the patient, the spring clip can be urged distally along the needle shaft by the necked portion of the sleeve pressing against the proximal wall of the spring clip until the proximal wall.

The perimeter defining the opening on the proximal wall can contact the change in profile on the needle, which stops further distal movement of the spring clip and further expansion of the sleeve.

The engagement between the change in profile and the opening in the proximal wall of the spring clip or the distal end of the sleeve can prevent the spring clip from displacing distally off of the needle.

The distally facing surface, such as the perimeter defining the opening, of the proximal wall can abut against the change in profile to prevent further distal movement of the spring clip following movement to the used or protected position in which the needle tip is shielded.

Because the change in profile can be larger in size than the opening, the spring clip cannot slide past the change in profile, thereby confining the needle tip within the spring clip and preventing unintended needle sticks after the needle 440 has been removed from the patient.

Because the aperture at the distal end of the sleeve is smaller than the spring clip, the spring clip can be confined within the interior cavity of the sleeve between the distal end of the sleeve and the necked portion.

The distal portion of the sleeve between the distal end of the sleeve and the necked portion can function as a housing having an interior cavity for accommodating the spring clip.

The housing can be sized and shaped to accept the spring clip therein and allow the resilient arms of the spring clip to move between the ready to use position and the protective position.

A safety needle assembly provided in accordance with further aspects of the present disclosure can comprise a catch extending from a side of a needle adapter engaging a securing device and functioning as a release button. The catch can be activated by a user to disengage the catch from the securing device to release the sleeve and allowing the compressed portion of the sleeve due to the engagement to expand.

Thus, the safety needle assembly can be manually activated to release the sleeve thereby allowing the sleeve and the spring clip inside the sleeve to cover the needle tip in the protective position. As such, it can be considered an active safety device.

The sleeve can move along the needle shaft as the needle is inserted into the patient.

The catch can extend from the needle adapter and form a generally L-shaped structure comprising a first segment extending from the needle adapter, a second segment, a flexible elbow coupling the second segment to the first segment, and a coupler at a free end of the second segment engaging the attach end of the securing device. In an example, the flexible elbow can be a living hinge. In another example, the flexible elbow can be a molded arcuate structure.

When the needle is inserted into the patient, the skin pushes against the distal end of the sleeve can to move the distal end of the sleeve, the spring clip, and the securing device proximally toward the catch, thereby further compressing the sleeve from its initial compressed state in the ready to use position. The securing device may be rigid and hence move the coupler as the securing device advances proximally on the needle, thereby causing the elbow to bend as the angle between the first segment and the second segment decreases.

The coupler can maintain contact with the attach end until the catch is activated by the user.

The shape of the coupler and the attach end is not limited and can embody shapes and means other than as shown.

The coupler can be a tab, a hook, a protrusion, or spline for engaging the attach end, which can be a complementary hook, lever, lip, notch, shoulder, spline, or combinations thereof.

The attach end can be ring-shaped and the coupler can form a curved resilient hook with a lip of the curved hook extending outwardly, such that the hook-shaped coupler can be squeezed inside a bore of the attach end to form an interference fit when engaged in the ready to use position.

The flexible elbow may be elastic and capable of storing elastic energy as the elbow bends from the moving the second segment towards the first segment. Said differently, as the angle formed between the first and second segments decrease, the potential elastic energy stored in the elbow may increase.

The elbow can provide a resistive force against the securing device to maintain a positive engagement between the coupler and the attach end as the securing device moves proximally towards the catch.

If the securing device is flexible instead of being rigid, the catch does not flex at the elbow as the distal end of the sleeve moves proximally while the sleeve compresses. Tension can be maintained in the securing device between the sleeve and the catch in the ready to use position and during use to maintain engagement between the coupler of the catch and the attach end of the securing device.

The distal end of the sleeve and consequently the spring clip inside the distal portion of the sleeve can be free to slide along the needle shaft as the needle is inserted into the patient and removed from the patient while maintaining tension in the securing device.

When the needle assembly is removed from the patient, the user can activate the catch to release the attach end from the coupler so that the sleeve can extend to move the spring clip to cover the needle tip.

The user can activate the catch by pressing on the first segment, the elbow, or the second segment. Alternatively, the catch can be activated by pressing the elbow against a surface such as a table top. Still alternatively, any part of the catch can be pressed by a user or a surface, such as a table top, to activate so as to separate the attach end from the coupler.

The sleeve can also move along the needle shaft as the needle is inserted into the patient. For example, as the needle is inserted into a patient, the skin can cause the sleeve to move. Because the sleeve is fixed at one end, movement of the sleeve causes the sleeve to compress and store potential energy.

The safety needle assembly can be activated during insertion of the needle into the patient. The activation can occur prior to removable of the needle from the patient. When activated during an injection, the sleeve and the spring clip inside the sleeve can cover the needle tip upon removal of the needle and the protective position.

The safety needle assembly can be activated during insertion of the needle into the patient by interacting the securing device and/or the catch with the sleeve so that the sleeve causes the activation to separate the coupler and the attach end.

The catch can extend from the needle adapter and forms a generally L-shaped structure comprising a first segment extending from the needle adapter, a second segment, a flexible elbow coupling the second segment to the first segment, and a coupler at a free end of the second segment engaging the attach end of the securing device.

The shape of the coupler and the attach end is not limited.

The coupler can be shaped as a tab, a hook, a protrusion, or spline for engaging the attach end, which can be a complementary hook, lever, lip, notch, shoulder, spline, or combinations thereof.

The attach end can be shaped as a ring or an open hook, and the coupler can form an L-shaped hook with the second segment, such that the coupler can extend into a bore of the attach end to maintain engagement with the attach end in the ready to use position.

The needle can be inserted into the patient, the distal end of the sleeve can push against the skin of the patient to move the distal end of the sleeve, the spring clip, and the securing device proximally toward the catch, thereby further compressing the sleeve from its initial compressed state in the ready to use position. The sleeve has an axially fixed end and a free end that can move. When the free end moves, the sleeve can compress or can compress to a further compressed position or state.

The securing device may be rigid enough to move the coupler by pushing against the second segment as the securing device advances proximally on the needle, thereby causing the elbow to bend as the angle between the first segment and the second segment decreases.

Simultaneously, as the angle between the first segment and the second segment decreases, the coupler may move away from out of the bore of the attach end until the coupler is fully disengaged from the attach end.

As the sleeve compresses further from its initial compressed state in the ready to use position to a further compressed state, a bulge or enlarged region of the sleeve grows radially outward along the length of the sleeve. Thus, when the sleeve compresses, the sleeve goes from a first length to a shorter second length and from a first diameter dimension to a second diameter dimension, which is larger than the first diameter dimension.

As the needle extends deeper into the patient, the outer dimension or diameter of the sleeve can increase as the distal end of the sleeve moves proximally to compress the sleeve.

As the outer dimension of the sleeve grows radially, the sleeve can push against the arm of the securing device until the attach end at the end of the arm is moved away from the coupler of the catch, thereby disengaging the coupler from the catch.

As the needle is retracted from the patient, the distal end of the sleeve may press against the skin of the patient until the needle is fully removed with the sleeve and the spring clip covers the needle tip in the protected position.

A needle tip assembly provided in accordance with aspects of the present disclosure can comprise a needle adapter having an opening at a proximal end and a distal end; a needle extending distally from the distal end of the needle adapter, the needle having a needle shaft and a needle tip at a distal end of the needle shaft; a spring clip slidably riding on the needle shaft and having a proximal wall with an opening for the needle shaft to pass therethrough, the spring clip urging against the needle shaft adjacent the needle tip in a ready to use position, and shielding the needle tip in a secured position; a spring having a proximal end coupled to the needle adapter and a distal end contacting the proximal wall of the spring clip; and a securing device coupled to a latch part of the spring and compressing a portion of the spring under tension in the ready to use position and decoupled from the latch part of the spring by proximal movement of the spring clip.

The needle can further comprises a change in profile adjacent the needle tip on the needle located distal of the proximal wall of the spring clip, the opening of the spring clip has a size smaller than a size of the change in profile to prevent removing the spring clip from the needle, and the distal end of the spring is adjacent the change in profile in the secured position.

The spring clip can urge against opposite sides of the needle shaft when not in the secured position.

The needle guard can further comprise a pair of resilient arms extending distally from the proximal wall, the resilient arms urging against opposite sides of the needle shaft, and the resilient arms shielding the needle tip in the secured position.

The spring can be a compression spring.

The distal end of the spring can be attached to the proximal wall of the spring clip.

The distal end of the spring can be pressing against the proximal wall of the spring clip.

The spring clip can be freely rotatable about the spring.

The securing device can comprise an attach end coupled to the latch part of the spring to secure the spring clip in the ready to use position.

The attach end can elastically move away from the spring when the attach end is decoupled from the spring.

The latch part of the spring can be pressing against the attach end.

The attach end can be decoupled from the spring when the latch part moves proximally away from the attach end.

The attach end can be decoupled from the spring when decoupler of the spring urges proximally against the attach end.

The latch part of the spring can be a coil and the decoupler of the spring is an adjacent coil.

The attach end can be hook-shaped.

The attach end can be triangular shaped.

The securing device can comprise more than one attach end.

The securing device can further comprise a pivot point proximal of the proximal end of the spring.

The pivot point can be coupled to the needle adapter.

The securing device can further comprise a mount coupled to the needle adapter, the pivot point coupling the attach end to the mount.

The securing device can further comprise an arm having one end pivoting about the mount and another end connected to the attach end.

Almost an entire length of the spring can be under compression by the securing device.

Less than half a length of the spring can be under compression by the securing device.

The securing device can further comprise a pivot point distal of the proximal end of the spring.

The pivot point can be mounted on a different part of the spring.

The pivot point can be a loop connected to the different part of the spring.

The spring can extend nearly an entire length of the needle shaft in the ready to use position.

A further aspect of the present disclosure can include a method of making a needle tip assembly which can comprise: extending a needle distally from a distal end of a needle adapter through a spring and an opening in a proximal wall of a spring clip, the needle adapter having an opening at a proximal end, the needle having a needle shaft and a needle tip at a distal end of the needle shaft; urging the spring clip against the needle shaft adjacent the needle tip in the ready to use position; coupling a proximal end of the spring to the needle adapter; contacting a distal end of the spring with the proximal wall of the spring clip; and compressing a portion of the spring under tension with a securing device coupled to a latch part of the spring in the ready to use position, the securing device decoupling from the latch part of the spring clip for shielding the needle tip in a secured position, the securing device decoupling from the latch part of the spring by proximal movement of the spring clip.

The needle can further comprises a change in profile adjacent the needle tip on the needle located distal of the proximal wall of the spring clip, the opening of the spring clip has a size smaller than a size of the change in profile to prevent removing the spring clip from the needle, and the distal end of the spring is adjacent the change in profile in the secured position.

The spring clip can urge against opposite sides of the needle shaft when not in the secured position.

The needle guard can further comprise a pair of resilient arms extending distally from the proximal wall, the resilient arms urging against opposite sides of the needle shaft, and the resilient arms shielding the needle tip in the secured position.

The spring can be a compression spring.

The distal end of the spring can be attached to the proximal wall of the spring clip.

The distal end of the spring can be pressing against the proximal wall of the spring clip.

The securing device can comprise an attach end coupled to the latch part of the spring to secure the spring clip in the ready to use position.

The attach end can be decoupled from the spring when the latch part moves proximally away from the attach end.

The attach end can elastically move away from the spring when the attach end is decoupled from the spring.

The latch part of the spring can be pressing against the attach end.

The attach end can be decoupled from the spring when decoupler of the spring urges proximally against the attach end.

The latch part of the spring can be a coil and the decoupler of the spring is an adjacent coil.

The attach end can be hook-shaped.

The attach end can be triangular shaped.

The securing device can comprise more than one attach end.

The securing device can further comprise a pivot point proximal of the proximal end of the spring.

The pivot point can be coupled to the needle adapter.

The securing device can further comprise a mount coupled to the needle adapter, the pivot point coupling the attach end to the mount.

The securing device can further comprise an arm having one end pivoting about the mount and another end connected to the attach end.

Almost an entire length of the spring can be under compression by the securing device.

Less than half a length of the spring can be under compression by the securing device.

The securing device can further comprise a pivot point distal of the proximal end of the spring.

The pivot point can be mounted on a different part of the spring.

The pivot point can be a loop connected to the different part of the spring.

The spring can extend nearly an entire length of the needle shaft in the ready to use position.

Aspects of the present disclosure can also include a safety needle assembly which can comprise a needle attached to a needle adapter, a spring mounted on the needle with a spring clip or tip protector, and a securing mechanism for maintaining at least a portion the spring in a compressed state in a ready to use position.

### BRIEF DESCRIPTION OF THE DRAWINGS

The various embodiments of the present safety needle assemblies now will be discussed in detail with an emphasis on highlighting the advantageous features. These embodiments depict the novel and non-obvious safety needle assemblies shown in the accompanying drawings, which are for illustrative purposes only. These drawings include the following figures, in which like numerals indicate like parts:

FIGS. 1A-1C depict an embodiment of a safety needle assembly with a passive safety system prior, during, and after activation; and

FIGS. 2A-2C depict another embodiment of a safety needle assembly with a passive safety system prior, during, and after activation.

FIGS. 3A-3C depict yet another embodiment of a safety needle assembly with a passive safety system prior, during, and after activation.

FIGS. 4A-4C depict another embodiment of a safety needle assembly with a passive safety system prior, during, and after activation.

FIG. 4D depict an embodiment of a securing device of the safety needle assembly of FIGS. 4A-4C.

FIG. 4E depict another embodiment of a securing device of the safety needle assembly of FIGS. 4A-4C.

FIGS. 5A and 5B depict another embodiment of a safety needle assembly with an active safety system before and after activation.

FIGS. 6A-6C depict another embodiment of a safety needle assembly with an active safety system prior, during, and after activation.

FIG. 7A depicts yet another embodiment of a safety needle assembly with a passive safety system before activation.

FIGS. 7B and 7C depict variations of how the catch may disengage from the sleeve of the safety needle assembly of FIG. 7A.

FIG. 7D depicts the safety needle assembly of FIG. 7A after activation.

### DETAILED DESCRIPTION

The following detailed description describes the present devices, apparatuses, systems, and methods with reference to the drawings. In the drawings, reference numbers label elements of the present embodiments. These reference numbers are reproduced below in connection with the discussion of the corresponding drawing features.

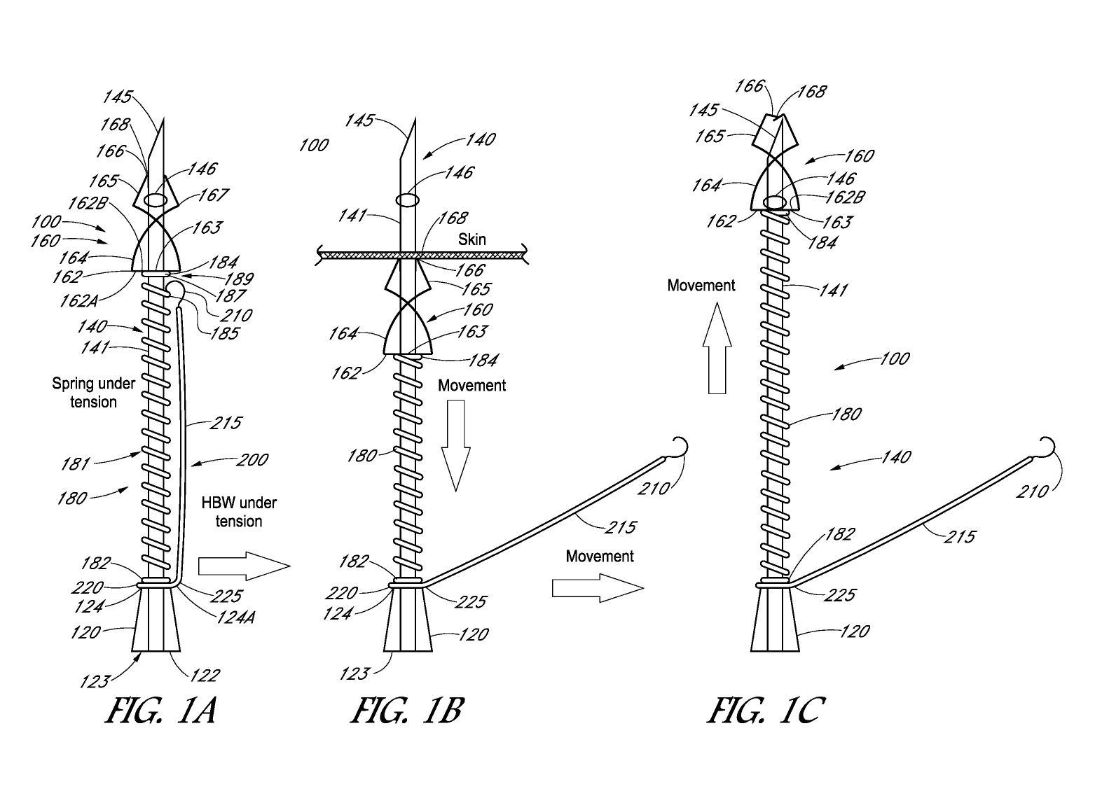

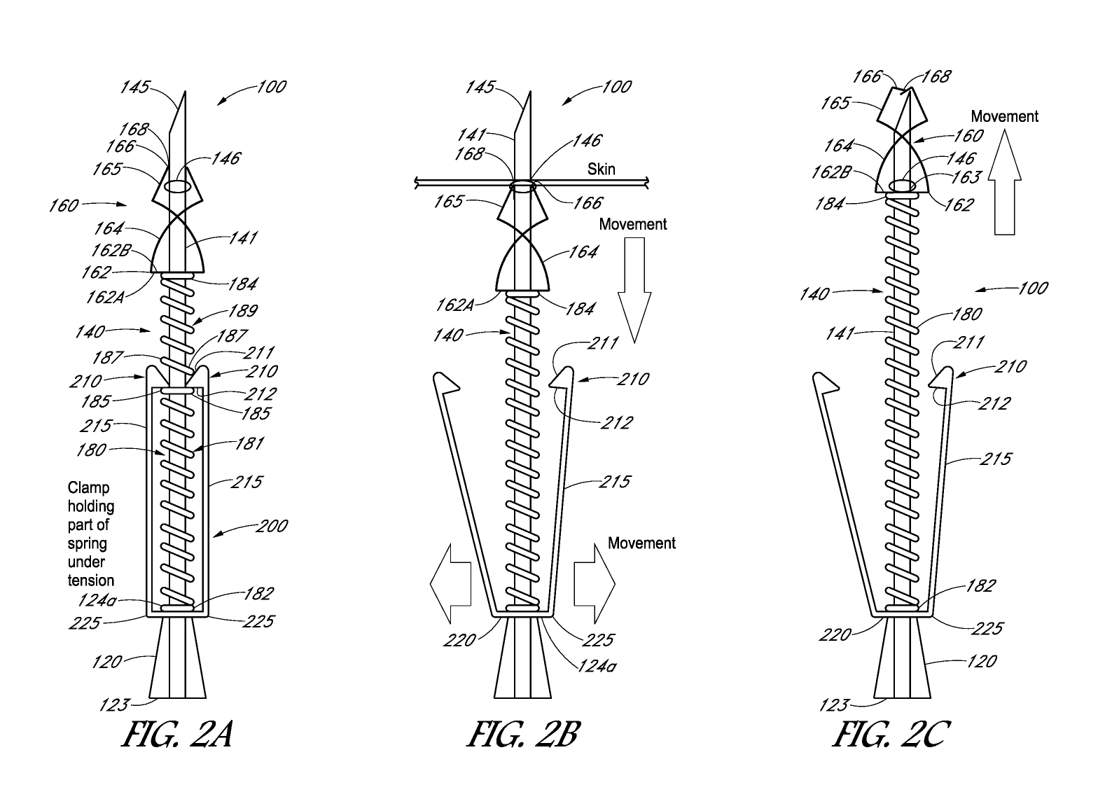

FIGS. 1A-1C illustrate an embodiment of a safety needle attachment, device, or assembly 100 provided in accordance with aspects of the present disclosure shown at various stages of operation. FIG. 1A illustrates an initial state of the safety needle assembly 100 in a ready to use position in which the needle tip of the needle is exposed, FIG. 1B shows the needle assembly during use, such as for performing an injection, and FIG. 1C shows the needle assembly after use in a protective position, such as being protected by a safety system.

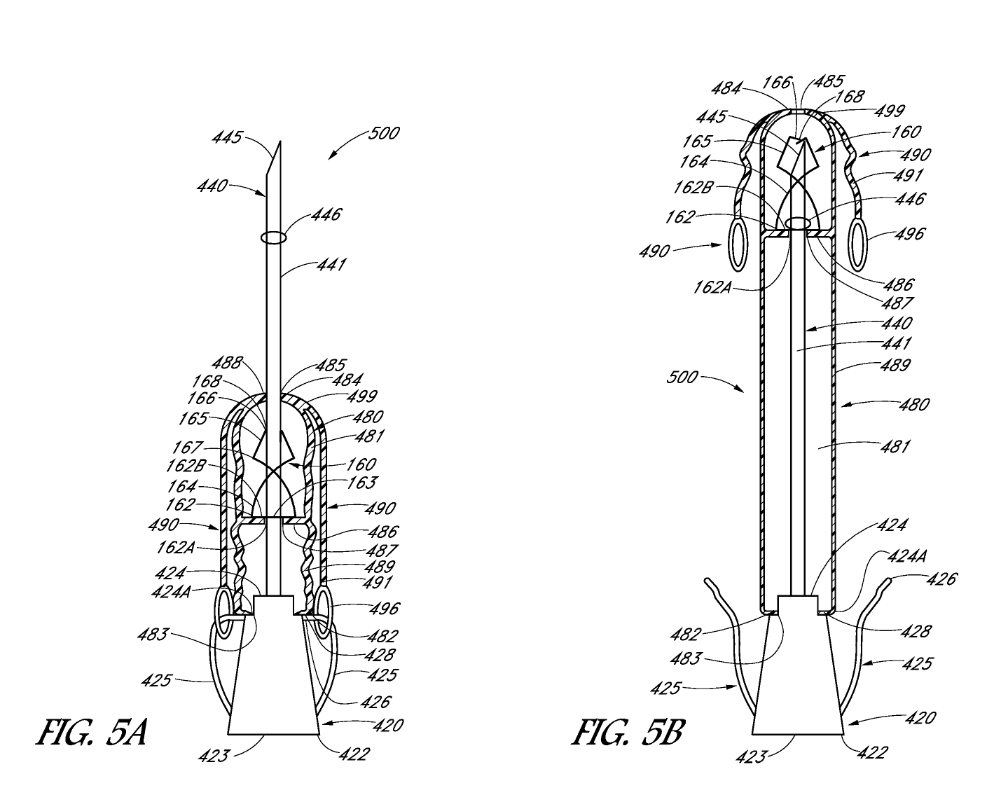

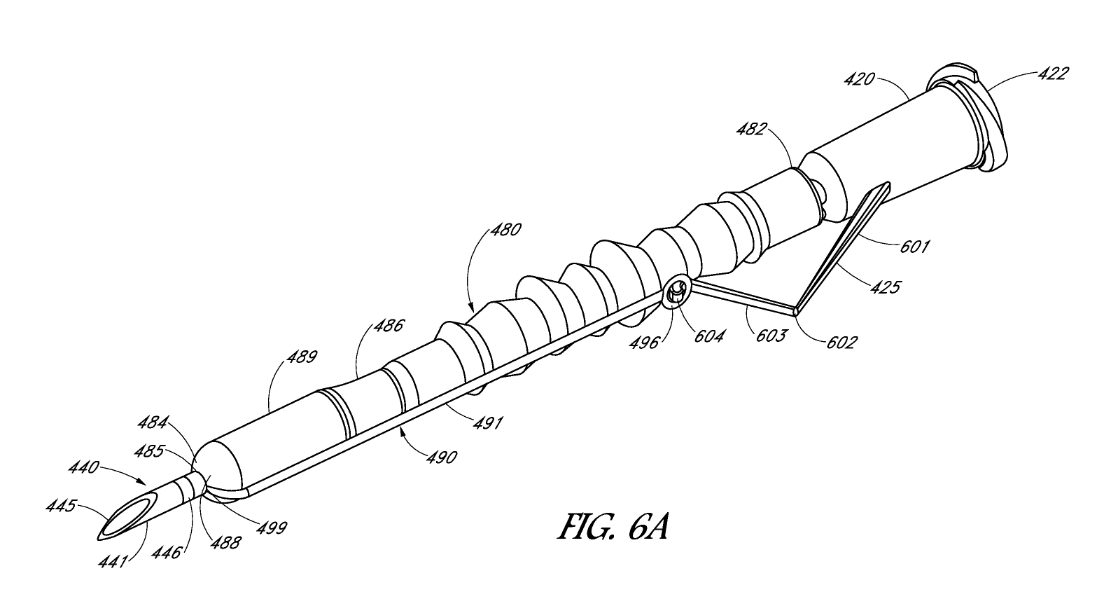

Referring now to FIG. 1A, the safety needle assembly 100 comprises a needle adapter 120, a needle 140, a needle guard, tip protector, or spring clip 160 slidably carried on the needle 140, a biasing member 180 positioned between the needle adapter 120 and the spring clip 160, and a securing device 200 compressing a portion of the biasing member 180 in a ready to use position. The biasing member 180 can be a spring, a spring with coils, or a sleeve with biasing features, such as an elastic sleeve capable of elastic deformation, as further discussed below with reference to FIGS. 4A-4F, 5A-5B, and 6A-6C. The needle adapter 120 has a proximal end 122 and a distal end 124 and may alternatively be referred to as a hub, such a needle hub. The needle 140 projects distally from the distal end 124 of the needle adapter 120. The proximal end 122 has an opening 123 for receiving a male medical implement such as a syringe for injecting fluid into the tissue of the patient or drawing fluid or blood from the patient. In some examples, the opening 123 is a female Luer for receiving a male Luer tip, such as a syringe tip. In other examples, the exterior of the needle adapter 120 has exterior threads for threaded engagement with a threaded collar on the syringe or other medical implements.

A ledge 124A is provided at the interface of the needle 140 and the distal end 124 of the needle adapter 120. The overall shape of the needle adapter 120 may vary and therefore vary the size and shape of the ledge 124A. In some examples, no material circular ledge is formed 124A and the distal end of the needle adapter 120 is approximately the size of the needle shaft. As shown, an outer surface of the needle adapter 120 tapers inward from the proximal end 122 to the distal end 124 to form a frustoconical shaped grip for a user to handily attach the safety needle assembly 100 to a syringe or other medical devices. A distal end of the frustoconical shaped grip has a ring shaped planar surface defining the ledge 124A for supporting a safety feature, as further discussed below. In some examples, a cross-section of the needle adapter 120 can be circular, rectangular, or any regular or irregular shape to form the grip. Surface features, such as fins and bumps, may be added to form a more effective gripping surface.

The needle 140 comprises a needle shaft 141 having a proximal end attached to the needle adapter 120, a needle tip 145 formed at a distal end of the needle shaft 141, and a change in profile 146 positioned adjacent the needle tip 145 on the needle shaft 141. The proximal end of the needle shaft 141 can be press fitted into the distal end 124 of the needle adapter 120 or glued into a glue well at the distal end 124 of the needle adapter 120. The needle tip 145 is configured to puncture the skin and tissue of a patient so that fluid can be injected into the patient, such as from a syringe attached to the needle adapter 120, or blood or other bodily fluid can be withdrawn through the needle tip 145 into the syringe. The needle tip 145 can be cut or ground to have any known prior art tip geometries, including a non-coring needle tip. The change in profile 146 has a cross-section larger than a cross-section of the needle shaft 141 to prevent the spring clip 160 from detaching from the safety needle assembly 100, as will be discussed below in reference to FIG. 1C. The change in profile 146 can be a crimp, a bulge, a sleeve, an attachment, or a material buildup.

A biasing member 180 is provided with the needle 140 to act as a passive safety device configured to move the spring clip or tip protector 160 in a distal direction to protect the tip from unintended needle sticks following use. In the illustrated embodiment, the biasing member 180 is a spring 180, such as a compression spring, having a plurality of interconnected coils between the proximal end 182 and the distal end 184 of the spring. The proximal end 182 of the spring 180 can be placed in abutting contact with the ledge 124a of the needle adapter 120. For example, the proximal end most coil of the spring 180 can be placed in abutting contact with the generally planar surface of the ledge 124a. To prevent the spring from separating from the needle adapter 120, the spring can be secured to the needle adapter, such as by mechanical inter-engagement, glue, bonding, or other securement means. In some examples, the proximal end 182 of the spring 180 can be attached directly to the needle shaft 141 at or near the interface of the needle 140 and the needle adapter 120. The distal end 184 of the spring 180 is attached to or pressed against the spring clip 160, or more specifically, to a proximally facing surface 162A of a proximal wall 162 of the spring clip 160 to propel the spring clip 160 distally towards the needle tip 145 to shield the needle tip from inadvertent needle sticks. In one example, the distal end 184 of the spring 180 presses against or abuts the spring clip 160 without mechanical inter-engagement so that the spring clip 160 can remain freely rotatable about the needle shaft 141 relative to the spring 180. In another example, the distal end 184 is attached to the proximally facing surface of the proximal wall 162 so that the spring clip 160 is not free to rotate about the needle shaft 141 relative to the spring 180. As shown in the figures, the spring 180 extends along nearly the entire length of the needle shaft 141 from the ledge 124a of the needle adapter 120.

Characteristics of the spring 180, such as the spring material, coil diameter, wire diameter or thickness, and pitch can be selected depending on the particular application of the safety needle assembly 100. For example, if the needle 140 is to extend deep into the patient, a low spring constant can reduce discomfort of the spring clip 160 pressing against the patient. A minimum spring constant may be achieved if the spring 180 can extend nearly an entire length of the exposed needle shaft 141 when the needle tip 145 is secured by the spring clip 160, as shown in FIG. 1C. That is, the longer the length of the spring 180 along the needle 140, the lower the spring constant can be achieved.

The spring 180 has a latch point 185 located between the proximal end 182 and the distal end 184. The latch point 185 can be a portion or a point of a coil for use by a securing device 200 to engage or latch onto. In some examples, the latch point 185 can be a tab, a hook, a protrusion, or an attachment extending outwardly from a coil of the spring 180 for the securing device 200 to latch onto. When the securing device 200 engages the latch point 185 of the spring 180, the spring 180 is divided between an uncompressed portion 189 distal of the latch point 185 and a compressed portion 181 proximal of the latch point 185. In some examples, the securing device 200 engages two latch points 185 on the coil along two spaced positions on the coil. The uncompressed portion 189 of the spring is defined between the latch point 185 and the distal end 184 of the spring, which can be contacted or abutted by a proximal wall of the spring clip 160. The compressed portion 181 of the spring is defined between the proximal end 182 and the latch point 185. As shown, nearly the entire length of the spring 180 is under compression or, put in another way, the uncompressed portion is zero or close to zero. In another example, at least 80% of the entire uncompressed length of the spring is under compression or, put in another way, the uncompressed portion is less than 20% of the entire length of the spring 180 between the spring clip 160 and the proximal end 182. In yet another example, at least 50% of the entire length of the spring 180 is under compression or, put in another way, the uncompressed portion of the spring is less than 50% of the entire length between the spring clip 160 and the proximal end 182.

The securing device 200, which may also be referred to as a hold back wire (HBW), can comprise a mount 220 disposed between the proximal end 182 of the spring 180 and the ledge 124a, an attach end 210 configured to engage with the latch point 185, and a securing arm 215 coupling the attach end 210 to the mount 220. In some examples, the mount 220 can be attached directly to the needle adapter 120 at a side of the ledge 124a so that the proximal end 182 of the spring 180 directly contacts the ledge 124a. As shown, the mount 220 has an opening for the needle 140 to extend therethrough and is pressed against the ledge 124a by the spring 180. In some examples, the mount 220 is pivotably connected to a coil of the spring 180 at or near the proximal end 182 of the spring 180.

The securing arm 215 can be pivotably attached to the mount 220 via a pivot point 225 as shown. The mount 220 can embody a coiled loop and unitarily formed with the securing arm 215 and the pivot point 225 can be a bend. In some examples, the securing arm 215 can be pivotably mounted directly to the needle adapter 120 without the need for a mount 220, such as being anchored directly into the wall surface of the needle adapter. The securing arm 215 normally extends at least partially radially outward from the needle 140 so that when the securing arm 215 is pivoted about the pivot point 225 and the attach end 210 engages the latch point 185, elastic potential energy is stored in the securing arm 215 and the pivot point 225. That is, in the normal state, the securing arm 215 and the attach end 210 at the free end of the securing arm are pointed away from the spring 180 and the needle 140. When the attach end 210 is engaged with the latch point 185 in the initial position, the securing arm 215 is simultaneously under tension caused by compressing the compressed portion 181 of the spring 180 and elastically deflected inwardly towards the spring 180 and the needle 140 caused by pivoting or bending the securing arm 215 towards the spring 180 to engage the attach end 210 with the latch point 185. Elastic potential energy is stored in the securing arm 215 and the pivot point 225 from the elastic deflection. The tension in the securing arm 215 caused by the spring 180 under compression maintains the engagement between the attach end 210 and the latch point 185 and prevents the securing arm 215 and the attach end 210 from elastically returning outwardly away from the spring 180.

In some examples, the attach end 210 can comprise a hook, a lever, a lip, a notch, a shoulder, a spline, or combinations thereof. The attach end 210 can removably engage the latch point 185 and upon compression of the spring 180 separates and deflects radially outwardly from the latch point 185, as further discussed below. The removable engagement can be a simple overlap with friction, a positive engagement with part of the attach end 210 entering part of the latch point 185 or combinations thereof.

The securing device 200 is coupled to the latch point 185 via the attach end 210 thereby dividing the spring 180 into the compressed portion 181 proximal of the latch point 185 and the uncompressed portion 189 distal of the latch point 185 in the ready to use position, as previously discussed. The location of the latch point 185 between the proximal and distal ends of the spring 180 can determine how far the spring clip 160 can travel in the proximal direction, such as when pressed against and pushed by the skin in the proximal direction during an injection, before the attach end 210 decouples from the latch point 185. As shown, the latch point 185 is located near a distal end 184 of the spring so a relatively small displacement of the spring clip 160 in the proximal direction can decouple the attach end 210 from the spring 180. In another example, if the latch point 185 is located near a proximal end 182 of the spring 180, then a greater displacement of the spring clip 160 is required before the attach end 210 is decoupled from the spring 180. Therefore, depending on the penetration depth of the needle 140 into the patient, the location of the latch point 185 and attach end 210 along the length of the spring 180 can be selected by the required proximal movement of the spring clip 160.

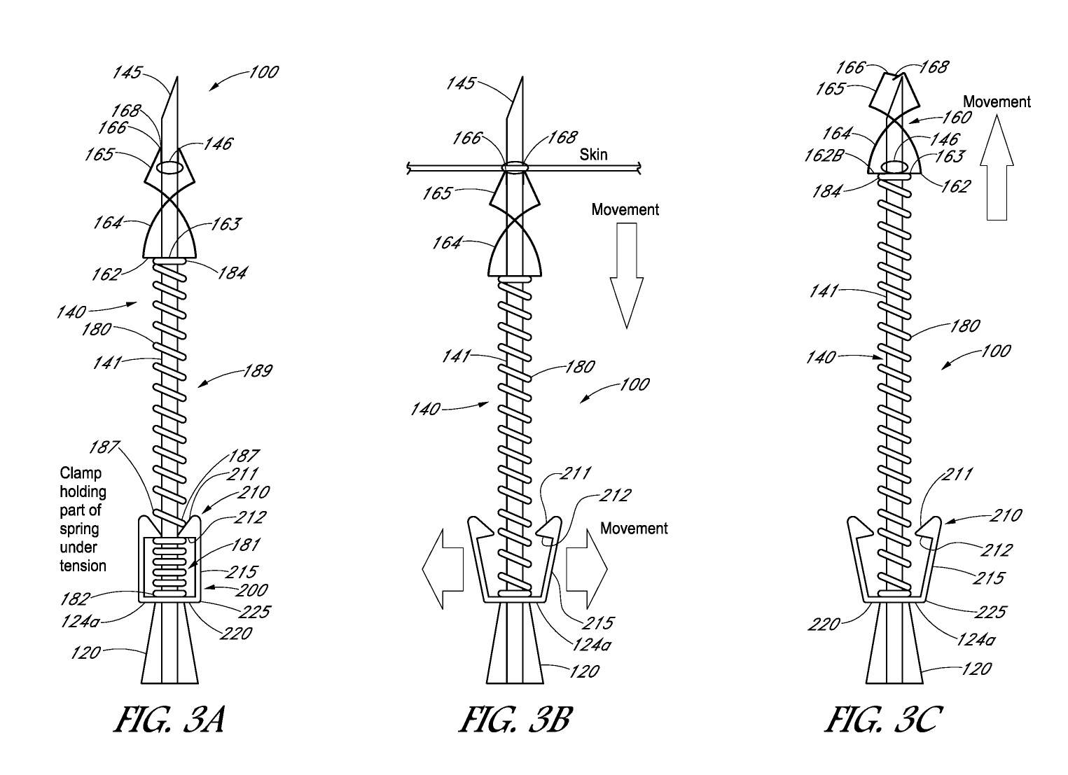

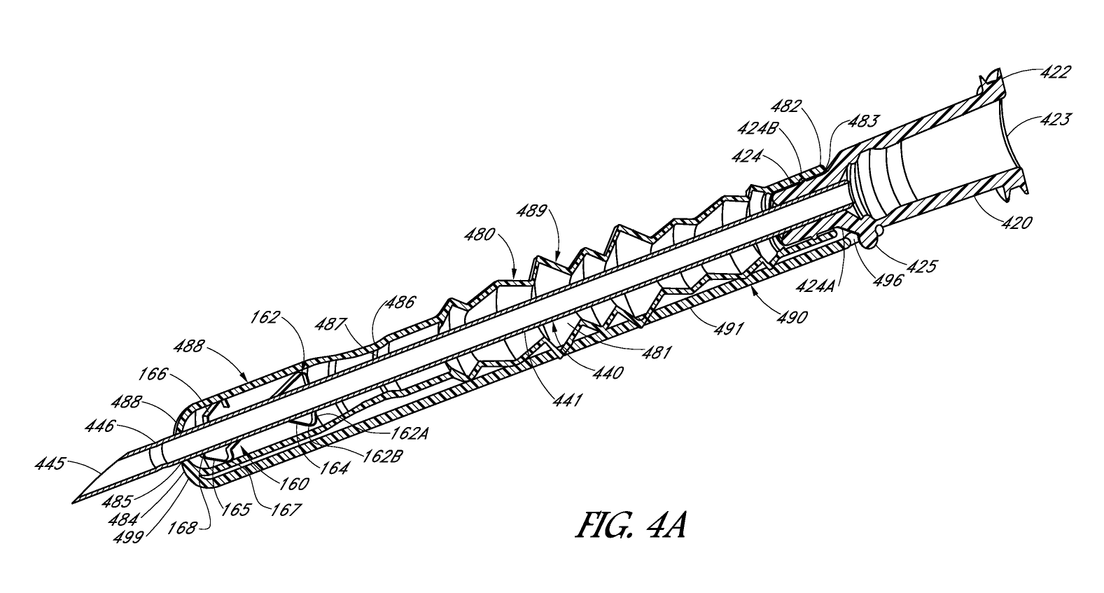

The attach end 210 is decoupled from the latch point 185 of the spring 180 by movement of the latch point 185 relative to the attach end 210 until contact between the latch point 185 and the attach end 210 is removed. In examples where a simple friction engagement is utilized, such as the embodiment of FIGS. 2A-2C, the attach end 210 can be moved relative to the latch point 185 to decouple the attach end from the latch point. In an example, a decoupler 187 can be incorporated to decouple the latch point 185 from the attach end 210. The decoupler 187 can press against the attach end 210 and exert at least a radially outwardly force or component forces to separate the contact with the latch point 185. As shown in FIGS. 1A and 1B, the decoupler 187 can be an adjacent coil immediately distal of the latch point 185. Thus, the decoupler 187 can contact and deflect the attach end 210 radially outwardly, can compress the coils of the spring to move the latch point 185 in a proximal direction away from the attach end 210, or both to separate the latch point 185 and the attach end 210 from one another. In some examples, the decoupler 187 can be an attachment or protrusion extending from the adjacent coil distal of the latch point 185. In other examples, the decoupler can be the spring clip 160 and the spring clip exerts a downwardly force or component forces to separate the latch point 185 and the attach end 210 from one another.

In an example, the decoupler 187 operates by contacting and pressing the attach end 210 outwardly from the latch point 185 until the attach end 210 is no longer engaged with the latch point 185. In some examples, the decoupler 187 presses against the latch point 185 and moves the latch point 185 proximally until the attach end 210 no longer engages the latch point 185. As shown in FIGS. 1A-1C, the attach end 210 can be shaped like a hook with the free end of the hook engaging with the latch point 185. Because the compressed portion 181 of the spring is held in a compressed state by the attach end 210 of the securing device 200, the safety needle assembly 100 is in an equilibrium state in the ready to use position of FIG. 1A.

With reference to FIG. 1B, the decoupler 187 is moved proximally when the needle 140 is inserted into the patient thereby moving the spring clip 160 proximally towards the latch point 185. As the spring clip 160 advances proximally on the needle, the uncompressed portion 189 starts to compress and the decoupler 187 advances towards the attach end 210. Further proximal movement causes the decoupler 187 to press against the attach end 210 and forces the attach end 210 to decouple or disengage from the latch point 185. Alternatively or in combination therewith, further proximal movement causes the compressed portion 181 of the spring to further compress, which moves the latch point 185 proximally away from the attach end 210. The securing arm 215 then elastically springs outwardly away from the spring 180 due to the stored elastic potential energy and remains out of the path of the spring 180 as shown in FIGS. 1B and 1C. Once the attach end 210 is no longer engaged with the latch point 185, the spring clip 160 can further move proximally depending on the depth of the needle insertion and the compressed spring 180 is ready to expand upon removal of the needle from the patient to move the tip protector or spring clip 160 over the needle tip, as shown in FIGS. 1B and 1C.

The spring clip 160 is prevented from moving distally by the securing device 200 compressing the compressed portion 181 of the spring 180 in the ready to use position. The spring clip 160 is configured to slide along the needle shaft 141 to shield the needle tip 145 from unintended needle pricks following activation, such as following use and the attach end is separated from the latch point. The spring clip 160 can slide proximally from the initial or ready to use position shown in FIG. 1A to an in-use position shown in FIG. 1B where the spring clip is further spaced from the needle tip, and then distally to the after-use, closed, or secured position shown in FIG. 1C where the spring clip is effectively located over the needle tip to shield the needle tip, such as after the needle 140 is removed from the patient.

As shown, one embodiment of the spring clip 160 of the present disclosure comprises a proximal wall 162 and two resilient arms 164 extending distally from the proximal wall 162. In one example, the spring clip 160 has a proximal wall 162 having a proximally facing surface 162A and a distally facing surface 162B. The proximal wall 162 can be generally circular, round or rectangular and can include a circular or non-circular aperture or perimeter defining an opening 163 for the needle 140 to extend therethrough. The opening 163 can be sized larger than the needle shaft diameter but smaller than a size of the change in profile 146 to prevent the proximal wall 162 from moving distally past the change in profile 146 in the secured position shown in FIG. 1C. In other examples, the spring clip 160 can embody a number of different guard devices or configurations for covering and securing the needle tip 145 from inadvertent needle sticks after withdrawing the safety needle following use. Exemplary spring clips 160 can be found in U.S. Pat. Nos. 6,616,630 and 8,827,965 and as the needle safety element presented in U.S. patent application Ser. No. 13/257,572, published as US 2012/0046620 A1, the contents of which are expressly incorporated herein by reference.

In the illustrated embodiment, each resilient arm 164 comprises a distal wall 165 having a free end 166 biased against diametrically opposite portions or sides of the needle shaft 141 when not in the secured position. As shown, the resilient arm 164 also has an elbow 167 extending from the wall 162 and a tip 168 at the free end 166 of the arm 164. The tip 168 can be a curved lip or hook which allows the free ends 166 to slide smoothly along the needle shaft 141. In some examples, the tips 168 can each have a simple straight edge with optional low friction material added to improve sliding relative to the surface of the needle, such as medical grade lubricant. The free ends 166 of the spring clip 160 are slidably positioned on opposite sides of the needle shaft 141 to allow the spring clip 160 to slide axially while minimizing any moment about the needle 140. As shown, the tips 168 of the two resilient arms 164 press against the needle shaft 141 in the ready to use position of FIG. 1A and the in-use position shown in FIG. 1B. The resilient arms 164 are elastically deflected outwardly by the needle shaft 141 and the ends 166 of the distal walls 165 are pressed against diametrically opposite points of the needle shaft 141.

Referring now to FIG. 1B, when the needle 140 is inserted into the patient, the spring clip 160 is moved proximally along the needle shaft 141 by abutting against the skin of the patient. As the needle 140 is inserted deeper into the patient, the spring clip 160 moves proximally thereby compressing the uncompressed portion 189 of the spring 180 and bringing the decoupler 187 closer to the latch point 185. Eventually, the decoupler 187 of the spring 180 presses against the attach end 210 or moves the latch point 185 proximally of the attach end 210 to disengage the attach end 210 from the latch point 185. Once the attach end 210 is no longer engaged with the latch point 185, the elastic securing arm 215 elastically springs outwardly away from the spring 180 and remains displaced from the spring 180. At this point, the spring clip 160 is pressed against the skin with the full length of the spring 180 unrestricted by the securing device 200.

Referring now to FIG. 1C, as the needle is 140 is removed from the patient, the spring clip 160 slides distally along the needle shaft 141 by the expansion force of the spring until the proximal wall 162, and more specifically the perimeter defining the opening 163 on the proximal wall, contacts the change in profile 146 on the needle, which stops further distal movement of the spring clip and further expansion of the spring. Before, concurrently, or shortly thereafter, the resilient arms 164 of the spring clip 160, now no longer pressing against the needle shaft 141, activate and move radially to move the two distal walls over the needle tip 145 to cover the needle tip 145 in the secured position. In the secured position, the free ends 166 or the tips 168 of the resilient arms 164 slide off the needle shaft and elastically move towards each other to cover the needle tip 145 to prevent inadvertent needle sticks. In some examples, the distal walls can each have folded tabs, such as two or more folded tabs, to define a holding space for capturing the needle tip therein to prevent the needle tip from moving laterally outside the side edges of the resilient arms 164. The resilient arms 164 can also have different lengths so that when the spring clip 160 is activated, the two distal walls 165 are axially offset and covering the needle tip.

The engagement between the change in profile 146 and the opening 163 in the proximal wall 162 of the spring clip 160 prevents spring clip 160 from displacing distally off of the needle. Specifically, the distally facing surface 162B, such as the perimeter defining the opening 163, of the proximal wall 162 abuts against the change in profile 146 to prevent further distal movement of the spring clip following movement to the used or protected position in which the needle tip is shielded. Because the change in profile 146 is larger in size than the opening 163, the spring clip 160 cannot slide past the change in profile, thereby confining the needle tip 145 within the spring clip 160 and preventing unintended needle sticks after the needle 140 has been removed from the patient.

In some examples, a cylinder or housing having a cavity for accommodating the spring clip 160 is contemplated. For example, the housing can resemble a cup having an elongated wall, one close end with an opening for the needle and one open end, which can be larger in size than the opening on the closed end. The housing can be placed at a distal end of the spring or somewhere near the distal end. The housing can be sized and shaped to accept the spring clip therein and allow the resilient arms of the spring clip to move between the ready to use position and the protective position, shown in FIG. 1C. The housing can have a distal end edge that extends distal of the spring clip 160 so that when the needle is used on a patient, the skin pushes against the distal end edge of the housing and not the spring clip 160. In some examples, the elongated wall can be a partially continuous, such as being semi-enclosed or having only part of a circumference. This housing can be practiced with any of the needle assemblies discussed elsewhere herein.

FIGS. 2A-2C illustrate another embodiment of a safety needle assembly 100 provided in accordance with further aspects of the present disclosure. The present safety needle assembly 100 is similar to the safety needle assembly of FIGS. 1A-1C with a few exceptions. In the present embodiment, the securing device 200 comprises two attach ends 210 and the configuration of the attach ends 210 is also different. Each attach end 210 of the present embodiment has an engaging surface 212 and a pressing surface 211 angled from the engaging surface 212. The securing arm 215 extends from the engaging surface 212 of each attach end 210 and can be pivotally attached to the mount 220 or the needle adapter 120 at the pivot point 225. As shown, the mount 220 is sandwiched between the proximal end 182 of the spring 180 and the ledge 124a of the needle adapter 120.

With reference now to FIG. 2A, each attach end 210 is engaged with a latch point 185 located on opposite sides of the spring 180 to secure the needle assembly in a ready to use position. In this position, the spring 180 has a compressed portion 181 between the latch points 185 and the proximal end 182 of the spring 180 and an uncompressed portion 189 between the latch point 185 and the spring clip 160. The position of the latch points 185 for the two attach ends 210 along the length of the spring are shown closer to the distal end 184 of the spring 180 than the proximal end 182 of the spring 180, but can be positioned anywhere along the length of the spring 180 depending on, as an example, the depth of needle insertion into the patient. Thus, the position of the latch points relative to the length of the spring is selectable depending on the desired needle application. In some examples, the latch points 185 can be axially offset to match with axially offset attach ends 210.

Decouplers 187 are incorporated and are located on an adjacent coil distal of the coil with the latch points 185. In one example, the decouplers are located on the same distal coil. In another example, the decouplers are located on different coils. In some embodiments, a multi-stage release system is contemplated with the latch points 185 staggered along the length of the spring 180 such that the securing arms 215 have unequal lengths to form a first and second compressed portions and one uncompressed portion. For example, a first latch point can be located closer to the distal end 184 of the spring 180 and a second latch point can be located between the first latch point and the proximal end 182 of the spring 180. In this example, the first compressed portion is defined between the first latch point and the second latch point, and the second compressed portion is located between the second latch point and the proximal end of the spring. In the first stage, the first decoupler disengages the first attach end from the first latch point so that the first compressed portion and the uncompressed portion combine to form a new uncompressed portion. In the second stage, the second decoupler disengages the second attach end from the second latch point to fully release the spring 180 from the securing device 200.

The shape of each of the attach ends 210 can also affect the required displacement of the decoupler, the spring clip, the housing for accommodating the spring clip, or all the above before separation between the release ends and the attach points. The shape and size of the attach end 210 can determine how far the decoupler 187 must travel before abutting and pushing the attach end and decoupling the attach end 210 from the latch point 185 of the spring 180. For example, if the pressing surface 211 is closer to the decoupler 187 of the spring 180 or had a different angle in the ready to use position as shown in FIG. 2A, then a smaller amount of movement of the spring clip 160 may release the attached points to free the compressed portion 181 of the spring 180 than if the pressing surface 211 is further proximally away from the decoupler 187. In some examples, the needle device can be practiced with a single attach end 210 for attaching to a single latch point. In other examples, there can be three or more attached ends for attaching to three or more attach points.

Referring now to FIG. 2B, when the needle 140 is inserted into the patient, the spring clip 160 is moved proximally towards the two latch points 185. As the spring clip 160 moves proximally towards the latch points, the decouplers 187 located on an adjacent coil distal of the latch points 185 strike the pressing surfaces 211 of the attach ends 210. Because the pressing surface 211 is angled with respect to the engaging surface 212, the proximal movement of the decoupler 187 pushes the attach end 210 outwardly away from the spring 180, due to component forces, until the engaging surface 212 is no longer engaging the latch point 185. At this point, the two securing arms 215 and the two attach end 210 elastically spring outwardly away from the spring 180 and release the compressed portion 181 of the spring 180. In some examples, two decouplers 187 are axially offset or staggered so that one pair of securing arm 215 and attach end 210 will release prior to the release of the second pair of securing arm 215 and attach end 210. Furthermore, if the latch points 185 are staggered or axially space, the first compressed portion on one side of the spring is released when the first decoupler disengages the first attach end from the first latch point and the second compressed portion on the opposite side of the spring is released when the second decoupler disengages the second attach end from the second latch point.

With reference now to FIG. 2C, as the needle 140 is withdrawn from the patient, the spring clip 160 is advanced distally by the spring 180 towards the needle tip 145 until the needle tip is covered by the spring clip 160. Distal movement of the spring clip 160 is stopped by the proximal wall engaging the change in profile near the needle tip, as previously discussed with reference to the first embodiment.

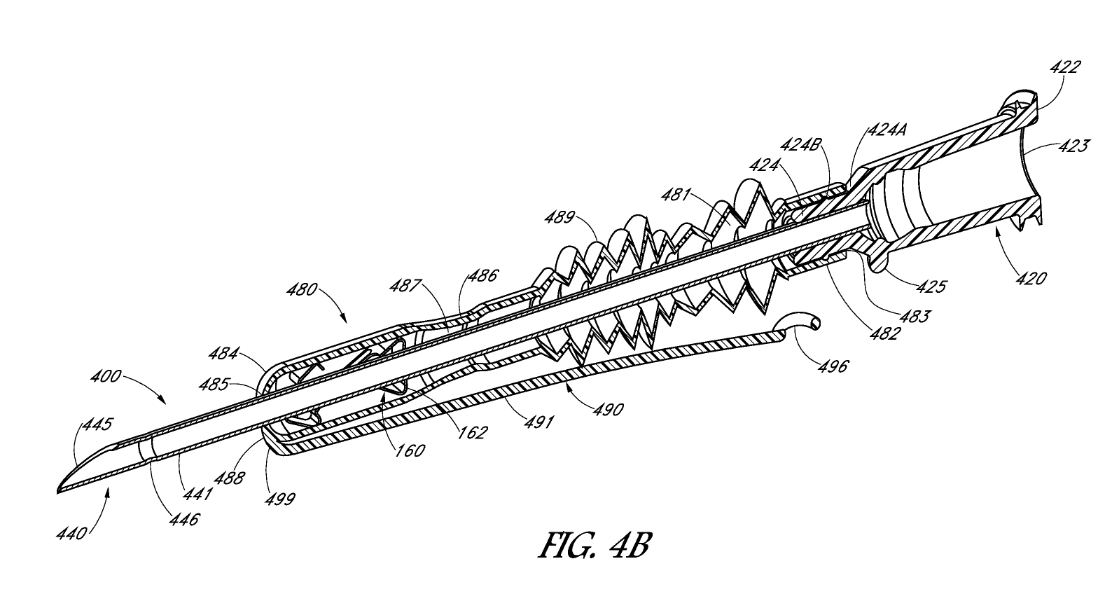

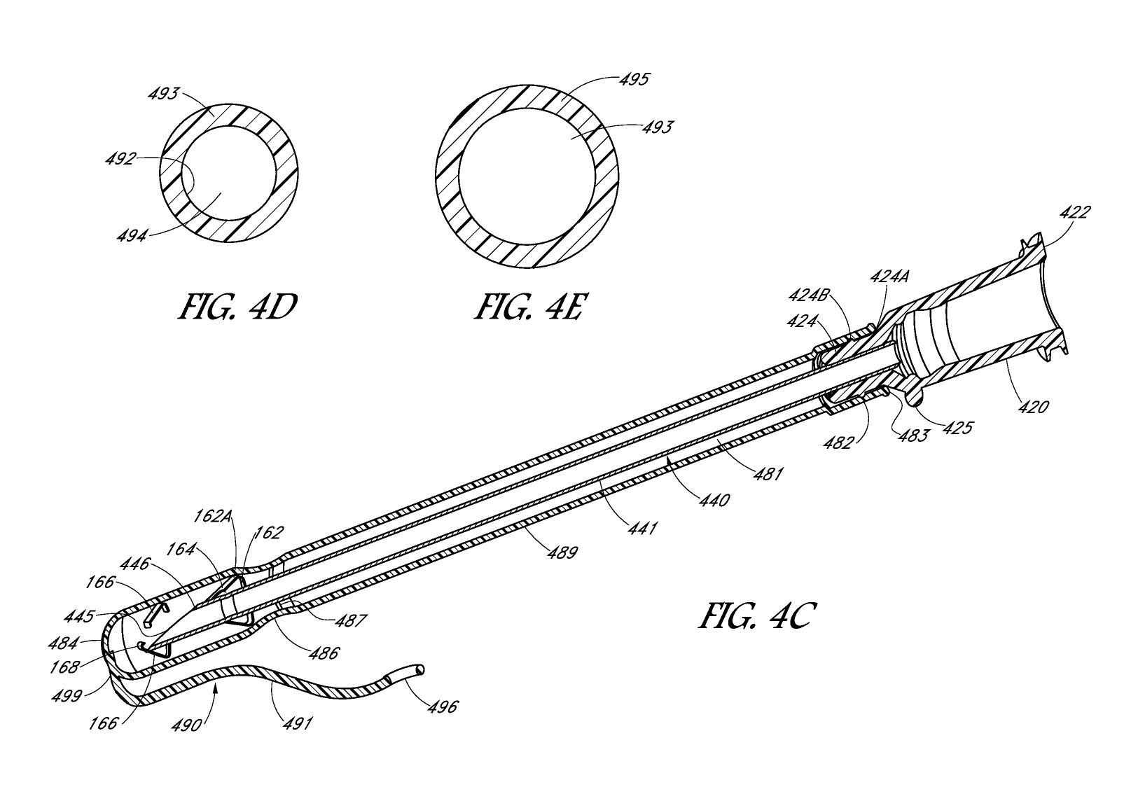

With reference now to FIGS. 3A-3C, the illustrated embodiment of the safety needle assembly 100 is similar to the safety needle assembly of FIGS. 2A-2C except that the attach ends 210 and the latch points 185 are located closer to the proximal end 182 of the spring 180 compared to the embodiment of FIGS. 2A-2C. In one example, the attach ends 210 can compress about half the length of the spring 180. In another example, the attach ends 210 can compress less than half the length of the spring 180. The longer the uncompressed portion 189 than the compressed portion 181, the longer the spring clip 160 can be displaced before the decouplers 187 disengage the attach ends 210 from the latch points 185.

Referring to FIG. 3A, the longer uncompressed portion 189 allows the spring clip 160 to travel proximally a longer distance before the attach end 210 releases the compressed portion 181 of the spring 180. This allows the needle 140 to penetrate deeper into the patient before the compressed portion 181 of the spring 180 is released.

Referring to FIG. 3B, when the needle 140 is inserted into the patient, the spring clip 160 moves proximally thereby compressing the uncompressed portion 189 of the spring 180. In the illustrated embodiment, because the uncompressed portion 189 is longer than the compressed portion 181, the amount of force required to move the spring clip 160 proximally is less than if the uncompressed portion 189 was shorter, assuming other spring characteristics being the same. The longer the uncompressed portion 189, the less the pressure applies by the spring clip 160 against the patient. Other factors, such as the spring constant of the spring 180 and the choice of material of the spring 180 can also affect the overall pressure of the spring clip 160 against the patient when inserting the needle into the patient. As the spring clip 160 is moved proximally towards the needle adapter 120 by pressing against the skin of the patient, the decoupler 187 pushes against the pressing surfaces 211 of the two attach ends 210 thereby forcing the attach ends 210 down and away from the latch points 185 of the spring 180 until both attach ends 210 are decoupled from the latch points 185 of the spring 180. As each attach end 210 is decoupled from the part of the spring 180, the securing arm 215 springs outwardly away from the spring 180.

Referring to FIG. 3C, as the needle 140 is removed from the patient, the spring clip 160 moves distally under spring force towards the needle tip 145 while providing resistance against the patient with decreasing force, which is typical of spring force characteristics of an expansion spring. When the needle 140 is completely removed from the patient, the ends 166 of the spring clip 160 move distal of the needle tip 145 and elastically spring back towards each other to cover the needle tip 145. The change in profile 146 prevents the spring clip 160 from further distal movement to prevent removing the spring clip 160 from the needle 140.

FIGS. 4A-4F illustrate an embodiment of a safety needle attachment, device, or assembly 400 provided in accordance with aspects of the present disclosure shown at various stages of operation. FIG. 4A illustrates an initial stage of the needle assembly 400 in a ready to use position in which the needle tip of the needle is exposed, FIG. 4B shows the needle assembly during use, such as for performing an injection, and FIG. 4C shows the needle assembly after use in a protective position, such as being protected by a safety system.

Referring initially to FIG. 4A, the safety needle assembly 400 can comprise a needle adapter 420, a needle 440, a needle guard or spring clip 160 slidably positioned on the needle 440, a biasing member 480 in the form of an elastic sleeve 480 enclosing the spring clip 160 and a portion of the needle 440 proximal of the needle tip, such as proximal of the spring clip, and a securing device 490 compressing at least a portion of the elastic sleeve 480 in a ready to use position. The needle adapter 420 has a proximal end 422 and a distal end 424 and may alternatively be referred to as a hub or needle hub. The needle 440 projects distally from the distal end 424 of the needle adapter 420. The proximal end 422 of the needle adapter has an opening 423 for receiving a male medical implement such as a syringe for injecting fluid into the tissue of the patient or drawing fluid or blood from the patient. In some examples, the opening 423 is a female Luer for receiving a male Luer tip, such as a syringe tip. In other examples, the exterior of the needle adapter 420 has exterior threads for threaded engagement with a threaded collar on the syringe or other medical implements, such as devices with a Luer lock.

A ledge 424A can be provided adjacent the distal end 424 of the needle adapter 420. The overall shape of the needle adapter 420 may vary and therefore the size and shape of the ledge 424A may also vary. In some examples, a ledge may be omitted. In other examples, the ledge 424A is a distal end 424 of the adapter. As shown, an outer surface of the needle adapter 420 can have portions that taper inward in the direction from the proximal end 422 to the distal end 424 to form an elongated enclosed cap, such as a partially cylindrical and partially frustoconical shaped structure for attaching the safety needle assembly 400 to a syringe or other medical devices.

Adjacent the distal end of the frustoconical shaped structure can be a ring shaped planar surface defining the ledge 424A for supporting the elastic sleeve 480, as further discussed below. In some examples, a cross-section of the needle adapter 420 can be circular, rectangular, or any regular or irregular shape structure, provided the interior is sized and shaped to receive a male medical implement, such a male Luer tip. The shape of the needle adapter 420 is not restricted and can vary depending on the needs and use of the safety needle assembly 400. Surface features, such as fins and bumps, may be added to form a more effective gripping surface on the exterior surface of the adapter.

A catch 425 can extend from the outer surface of the needle adapter 420. In one example, the catch can be located between the proximal end 422 and the distal end 424 but can be located at one of the ends. The securing device 490 can extend from an outer surface of the sleeve 480 and engage the catch 425 to compress a portion of the sleeve 480 to expose the needle tip 445 in the ready to use position, as further discussed below. Thus, the length of the securing device should be selected so that when engaged, it compresses at least part of the sleeve. Alternatively, the securing device 490 can be provided on the outer surface of the needle adapter 420 to engage a catch 425 extending from the outer surface of the sleeve 480 in the ready to use position.

The needle 440 comprises a needle shaft 441 having a proximal end attached to the needle adapter 420, a needle tip 445 at a distal end of the needle shaft 441, and, optionally, a change in profile 446 positioned adjacent the needle tip 445 on the needle shaft 441, such as proximal of the needle tip. The proximal end of the needle shaft 441 can be press fitted into the distal end 424 of the needle adapter 420 and/or glued into a glue well at the distal end 424 of the needle adapter 420. The needle tip 445 is configured to puncture the skin and tissue of a patient so that fluid can be injected into the patient, such as from a syringe attached to the needle adapter 420. Alternatively blood or other bodily fluid can be withdrawn through the needle tip 445 into the barrel of the syringe. The needle tip 445 can be cut or ground to have any known prior art tip geometries, including a non-coring needle tip. The optional change in profile 446 has a cross-section larger than a cross-section of nominal diameter of the needle shaft 441 to prevent the spring clip 160 from detaching from the safety needle assembly 400, as will be discussed below in reference to FIG. 4C. The optional change in profile 446 can be a crimp, a bulge, a sleeve having a bore mounted around the needle shaft, an attachment, or a material buildup.

The present safety needle assembly 400 can be considered a passive safety device in that the elastic sleeve 480 is configured to move the spring clip or tip protector 160 in a distal direction to protect the needle tip 445 from unintended needle sticks following use. The elastic sleeve can also prevent or reduce possible blood exposure by surrounding the needle shaft, which can be coated with blood or other bodily fluids following use.

In the illustrated embodiment, the elastic sleeve 480 is a hollow tubular structure defining an interior cavity 481 having an opening 483 at a proximal end 482 and a partially enclosed distal end 484 with an aperture 485 for the needle 440 to extend therethrough in the ready to use position. The sleeve 480 can surround or encase the needle 440 and the spring clip 160 with the needle tip 445 extending through the opening 483 and the aperture 485 surrounding the distal end 424 of the needle adapter 420.

To prevent the elastic sleeve 480 from separating from the needle adapter 420, the proximal end 482 of the elastic sleeve 480, such as at the ledge 424A of the needle adapter 420, can be coupled to the needle adapter 420 by mechanical inter-engagement, glue, bonding, welding, ultrasonic welding, or other securement means. In some examples, the proximal end 482 of the sleeve 480 can be attached directly to the needle shaft 441 at or near the interface of the needle 440 and the needle adapter 420.

A necked portion 486 can be formed on the sleeve 480 adjacent the distal end 484 of the sleeve 480 and proximal of the spring clip 160. The necked portion 486 may be a portion of the sleeve 480 that extends inwardly towards the needle 440 to form a reduced passage 487, which has a size or diameter less than that of the interior cavity 481 of the sleeve 480 to confine the spring clip 160 in the interior cavity 481 between the distal end 484 of the sleeve 480 and the necked portion 486. The necked portion 486 may also press against the spring clip 160, or more specifically, to a proximally facing surface 462A of a proximal wall 462 of the spring clip 160, to propel the spring clip 160 distally towards the needle tip 445 to shield the needle tip from inadvertent needle sticks, as further discussed below in reference to FIG. 4C. Thus, the reduced passage 487 can be sized to be large enough for the needle shaft 441 to pass through but small enough to advance the spring clip 160 along the needle shaft 441. Thus, the size or diameter of the passage 487 can be slightly larger than the needle shaft 441 and less than the proximal wall 162 of the spring clip 160. The passage 487 may also be the same size or slightly smaller than the needle shaft 441. The necked portion 486 may extend a short distance, as shown, or extend to the proximal end 482 of the sleeve 480. In one example, the necked portion 486 may be an internal shoulder extending radially inwardly from a surface of the interior cavity 481 of the sleeve 480 towards the needle 440 thereby forming the passage 487, which is also large enough for the needle shaft 441 to pass through but small enough to abut and advance the spring clip 160 along the needle shaft 441. The internal shoulder can be disc-shaped with an orifice as the passage 387 extending through the center for the needle 440 to extend and slide through.

In one example, the necked portion 486 of the sleeve 480 presses against or abuts the spring clip 160 but not inter-engage the spring clip so that the spring clip 160 can remain freely rotatable about the needle shaft 441 relative to the sleeve 480. In another example, the necked portion 486 inter-engages with the proximally facing surface 162A of the proximal wall 162 so that the spring clip 160 is not free to rotate about the needle shaft 141 relative to the sleeve 480. As shown in the figures, the sleeve 480 extends along nearly the entire length of the needle shaft 441 from the ledge 424a of the needle adapter 420.

The sleeve 480 can functions as a spring and has resilient properties that can generate a biasing force. The sleeve can surround the spring clip 160 and at least part of the needle 440. The sleeve can have spaced rings so as to form a uniform compressive body or can be allowed to compress and form random folds. The characteristics of the sleeve 480, such as the sleeve material and elastic properties of the sleeve 480, can be selected depending on the particular application of the safety needle assembly 400. For example, when a force is applied to the sleeve 480 to compress the sleeve 480 some distance, elastic energy is stored in the sleeve 480. When the force is removed, the sleeve 480 can elastically return to its original shape. The applied force required to compress the sleeve 480 may be proportional to the distance compressed by a spring rate, which can be linear or non-linear. A linear spring rate is a spring that has a spring constant that is directly proportional to the applied force.

A sleeve 480 having a high spring constant requires a larger applied force than a sleeve 480 having a smaller spring constant to compress the sleeve 480. For example, if the needle 440 is to extend deep into the patient, a low spring constant can reduce discomfort caused by the force of the sleeve 480 pressing against the patient. A minimum spring constant may be achieved if the sleeve 480 can extend nearly an entire length of the exposed needle shaft 441 when the needle tip 445 is secured by the spring clip 160, as shown in FIG. 4C. That is, the longer the length of the sleeve 480 along the needle 140, the lower the spring constant can be achieved.

The securing device 490, which may also be referred to as a hold back strap, can comprise an arm 491 extending from the outer surface of the sleeve 480 and an attach end 496 at a free end of the arm 491. The arm 491 and the attach end 496 can be integrally formed with the sleeve 480 or attached to the sleeve by mechanical inter-engagement, glue, bonding, welding, or other securement means. In the example shown, the arm 491 of the securing device 490 extends in the proximal direction from the sleeve 480. The arm 491 of the securing device 490 can also extend radially outwardly with respect to the needle axis from the sleeve 480 to ensure the attach end 496 at the free end of the arm 491 is moved away from the catch 425 to prevent the attach end 496 from reengaging the catch 425 when the attach end 496 disengages from the catch 425.

Referring to FIG. 4D, the arm 491 can be hollow and have a bore 492 extending through a body 493 of the arm 491 between the attach end 496 and the sleeve 480, and an elastic rod 494 inserted inside the bore to ensure the arm 491 extends at least partially radially outwardly when not engaged to the adapter, such as when not secured to the catch 425. Alternatively, referring to FIG. 4E, the arm 491 can comprise an outer resilient frame or sleeve 495 sized and shaped to fit over the arm 491 to extend the arm at least partially radially outwardly when not engaged to the catch. When the alternative resilient frame or sleeve is used, the arm 491 can be solid and without a bore.

Referring back to FIG. 4A, the attach end 496 engages the catch 425 on the needle adapter 420 when the safety needle assembly 400 is in the ready to use position, in which the needle tip extends out the needle guard and the sleeve and the device is ready for use. When the sleeve 480 is compressed a certain distance during use from the ready position, the attach end 496 can disengage from the catch 425 and spring radially outwardly to its natural state from the release of the stored elastic energy in the arm 491, as further discussed below with reference to FIG. 4B.

The arm 491 can extend outwardly radially from the outer surface of the sleeve 480 adjacent to or at the distal end 484 of the sleeve 480. As shown, the arm 491 extends outwardly from the sleeve 480 adjacent to the distal end 484 of the sleeve 480. In another example, the arm 491 can extend outwardly from the sleeve 480 adjacent to the distal end 484 by the necked portion 486 of the sleeve 480. The location of the attached end of the arm 491 along the length of the sleeve 480 can determine the amount of force applied to the sleeve 480 and the amount of compression required before the attach end 496 disengages from the catch 425.

The catch 425 can be a tab, a hook, a protrusion, or an attachment extending outwardly from the needle adapter 420 to present a surface for the securing device 490 to latch onto. The catch 425 can be located between the proximal end 422 and the distal end 424 of the needle adapter 420 or at one of the ends. In a specific example, the catch 425 can be located proximal of the ledge 424A. Alternatively, the catch 425 can be located on the sleeve 480 proximal of the arm 491 and the attach end 496.

When the attach end 496 of the securing device 490 engages the catch 425, the elastic sleeve 480 is at least partially compressed to provide tension along the securing device 490 to maintain the attach end 496 to the catch 425, as further discussed below. In some embodiments, depending on the location of where the arm 491 extends from the sleeve 480, the sleeve 480 may be compressed only at certain portions along the sleeve 480. More specifically, when the securing device 490 engages the catch 425, the sleeve 480 may be divided between an uncompressed portion 488 distal of an anchor point 499, which can be defined as where the arm 491 extends from the sleeve 480, and a compressed portion 489 proximal of the anchor point 499. The uncompressed portion 488 of the sleeve 480 can be located between the anchor point 499 and the distal end 484 of the sleeve 480.

The uncompressed portion 488 can be partially compressed when the needle 440 is inserted into the patient. In one example, the uncompressed portion 488 can be incompressible so that the uncompressed portion 488 would not squeeze the spring clip 160 inside the uncompressed portion 488 when the needle is inserted into a patient. Thus, the anchor point 499 can be located at or near the necked portion 486 of the sleeve 480 so that only the portion of the sleeve 480 proximal of the anchor point 499 compresses in the ready to use position. The compressed portion 489 of the spring can be defined between the proximal end 482 of the sleeve 480 at the ledge 424A and the anchor point 499. In one example, nearly the entire length of the sleeve 480 proximal of the spring clip 160 can be under compression. In another example, about 80% of the entire uncompressed length of the sleeve 480 can be under compression or, put in another way, the uncompressed portion can be less than 20% of the entire length of the sleeve 480. In yet another example, at least 50% of the entire length of the sleeve 480 can be under compression or, put in another way, the uncompressed portion of the spring can be less than 50% of the entire length of the sleeve 480.

The securing device 490 may normally extend at least partially radially outwardly with respect to the axis of the needle 140 when not engaged so that when the attach end 496 of the securing device 490 engages the catch 425, elastic potential energy is stored in the securing device 490. That is, in the normal state, the arm 491 and the attach end 496 at the free end of the arm 491 are pointed away from the sleeve 480 and the needle 440, so that when the attach end 496 is engaged with the catch 425 in the initial position or ready to use position, the arm 491 is simultaneously under tension caused by compressing the compressed portion 489 of the sleeve 480 and elastically deflected inwardly towards the sleeve 480 and the needle 440 caused by pivoting or bending the arm 491 towards the sleeve 480 to engage the attach end 496 with the catch 425. Elastic potential energy is stored in the arm 491 from the elastic deflection. The tension in the arm 491 caused by the sleeve 480 under compression maintains the engagement between the attach end 496 and the catch 425 and prevents the arm 491 and the attach end 496 from elastically moving away from the sleeve 480. Thus, the sleeve 480 can be maintained under compression in the ready to use position by the securing device 490, which can function as a hold back strap.

In some examples, the attach end 496 can comprise a loop, a hook, a lever, a lip, a notch, a shoulder, a spline, or combinations thereof. The attach end 496 can removably engage the catch 425 and upon compression of the sleeve 480, separate and deflect radially outwardly from the catch 425, as further discussed below. The removable engagement can be a simple overlap with friction, a positive engagement with part of the catch 425 entering part of the attach end 496, or combinations thereof. The catch 425 can be a tab, a hook, a protrusion, a notch, or an attachment angled at least partially proximally so that the attach end 496 can slip off easily when the sleeve 480 is further compressed when the needle 440 is inserted into the patient.

The securing device 490 is coupled to the catch 425 via the attach end 496 thereby dividing the sleeve 480 into the compressed portion 489 proximal of the anchor point 499 and the uncompressed portion 488 distal of the anchor point 499 in the ready to use position, as previously discussed. The location of the anchor point 499 between the proximal and distal ends of the sleeve 480 can determine how far the sleeve 480 and/or the spring clip 160 in the sleeve 480 can travel in the proximal direction, such as when the distal end 484 of the sleeve 480 is pressed against and pushed by the skin in the proximal direction during an injection. In one example, the anchor point 499 can be located near a distal end 484 of the sleeve 480 adjacent to the necked portion 486, so that a relatively small displacement of the sleeve 480 and/or the spring clip 160 in the proximal direction can decouple the attach end 496 from the sleeve 480. In another example, the anchor point 499 is located closer to a proximal end 482 of the sleeve 480 so that a greater displacement of the spring clip 160 is required before the attach end 496 can uncouple from the sleeve 480. Therefore, depending on the penetration depth of the needle 440 into the patient, the location of the anchor point 499 along the length of the sleeve 480 and the distance required to disengage the attach end 496 from the catch 425 can be selected by the required proximal movement of the spring clip 160.

The attach end 496 can decouple or uncouple from the catch 425 by movement of the attach end 496 relative to the catch 425 until contact between the catch 425 and the attach end 496 is removed. As shown in FIGS. 4A-4C, the attach end 496 can shape like a closed loop receive the catch 425 through the loop. In other examples, the attach end 496 can shape like a hook and the catch 425 have a closed loop or a ledge without or with a lip extending in a direction opposite the hook-shaped attach end 496 to positively engage the attach end 496 in the ready to use position. The compressed portion 489 of the sleeve 480 is held in a compressed state by the engagement of the attach end 496 with the catch 425.

With reference to FIG. 4B, the securing device 490 is moved proximally when the needle 440 is inserted into the patient and the distal end 484 of the sleeve 480 is moved against the skin to compress the sleeve and move the spring clip 160 proximally towards the catch 425. As the distal end 484 of the sleeve 480 and the spring clip 160 advance proximally on the needle 440, the uncompressed portion 488 compresses, if compressible, and the attach end 496 advances distally away from the catch 425 thereby breaking contact with the catch 425 and elastically springing outwardly away from the sleeve 480 due to the stored elastic potential energy. Once moved away from the catch, the attach end thereafter remains out of reach of the catch 425, as shown in FIGS. 4B and 4C. The sleeve 480 and the spring clip 160 inside the sleeve 480 can further move proximally even after the attach end separates from the catch depending on the depth of the needle insertion. With stored potential energy, the compressed sleeve is ready to expand upon removal of the needle 440 from the patient, which will move the tip protector or spring clip 160 distally as it expands to cover the needle tip 445, as shown in FIG. 4C.

The spring clip 160 is prevented from moving distally off of the needle tip by the constraint of the sleeve 480. Additionally, the opening on the proximal wall of the needle guard can engage the change in profile near the needle tip to limit distal movement of the spring clip or tip protector 160.

The spring clip or tip protector 160 is configured to slide along the needle shaft 441 to shield the needle tip 445 from unintended needle pricks following activation and upon expanding of the sleeve following use and the attach end 496 is separated from the catch 425. The spring clip 160 can slide proximally from the initial or ready to use position shown in FIG. 4A to an in-use position shown in FIG. 4B, where the spring clip 160 is further spaced from the needle tip 445, and then sliding distally to the after-use, closed, or secured position shown in FIG. 4C, where the spring clip 160 is located over the needle tip 445 to shield the needle tip 445, such as after the needle 440 is removed from the patient.

As shown, one embodiment of the spring clip 160 of the present disclosure comprises a proximal wall 162 and two resilient arms 164 extending distally from the proximal wall 162, as discussed above for FIGS. 1A-1C. The tips 168 of the free ends 166 of the two resilient arms 164 press against the needle shaft 441 in the ready to use position of FIG. 4A and in the use position shown in FIG. 4B. The resilient arms 164 are elastically deflected outwardly by the needle shaft 141 and the free ends 166 of the distal walls 165 are pressed against opposite points of the needle shaft 141.

Referring back to FIG. 4B, when the needle 140 is inserted into the patient, the distal end of the housing, which is the distal end 484 of the sleeve 480, pushes against the skin of the patient. The spring clip 160 is moved proximally along the needle shaft 441 by abutting against the inside surface of the interior cavity 481 of the sleeve 480 at the distal end 484 of the sleeve 480. As the needle 440 is inserted deeper into the patient, the spring clip 160 is pushed proximally along the needle shaft 441 by the distal end 484 of the sleeve 480 while further compressing the compressed portion 489 of the sleeve 480 to move the attach end 496 proximally and away from the catch 425. Eventually, the attach end 260 disengages from the catch 425 as the needle 440 extends deeper into the patient, at which time the arm 491 and the attach end 496 elastically spring outwardly away from the catch 425 and remain displaced from the catch 425. At this point, the distal end 484 of the sleeve 480 is pressed against the skin with at least part of the sleeve 480 under compression.

Referring now to FIG. 4C, as the needle 440 is removed from the patient, the spring clip 160 is urged distally along the needle shaft 441 by the necked portion 486 of the sleeve 480 pressing against the proximal wall 162 of the spring clip 160 until the proximal wall 162, and more specifically the perimeter defining the opening 163 on the proximal wall 162, contacts the change in profile 446 on the needle 440, which stops further distal movement of the spring clip 160 and further expansion of the sleeve 480. Before, concurrently, or shortly thereafter, the resilient arms 164 of the spring clip 160, now no longer pressing against the needle shaft 441, activate and move radially to move the two distal walls over the needle tip 445 to cover the needle tip 445 in the secured position. In the secured position, the free ends 166 of the resilient arms 164 slide off the needle shaft 441 and elastically move towards each other to cover the needle tip 445 to prevent inadvertent needle sticks. In some examples, the distal walls can each have folded tabs, such as two or more folded tabs, to define a holding space for capturing the needle tip therein to prevent the needle tip from moving laterally outside the side edges of the resilient arms 164. The resilient arms 164 can also have different lengths so that when the spring clip 160 is activated, the two distal walls 165 are axially offset and covering the needle tip 445. If no change in profile 446 on the needle 440 is provided, when the sleeve 480 is fully expanded, the length of the sleeve is selected to provide the constraint to stop further distal movement of the spring clip 160 when the spring clip 160 is activated and covers the needle tip 445.

The engagement between the change in profile 446 and the opening 163 in the proximal wall 162 of the spring clip 160 or the distal end 484 of the sleeve prevents the spring clip 160 from displacing distally off of the needle 440. Specifically, the distally facing surface 162B of the proximal wall, such as the perimeter defining the opening 163 of the proximal wall 162, abuts against the change in profile 446 to prevent further distal movement of the spring clip 160 following movement to the used or protected position in which the needle tip is shielded. Because the change in profile 446 is larger in size than the opening 163, the spring clip 160 cannot slide past the change in profile, thereby confining the needle tip 445 within the spring clip 160 and preventing unintended needle sticks after the needle 440 has been removed from the patient. Similarly, because the aperture 485 at the distal end 484 of the sleeve 480 is smaller than the spring clip 160, the spring clip 160 is confined within the interior cavity 481 of the sleeve 480 between the distal end 484 of the sleeve 480 and the necked portion 486. The distal portion of the sleeve 480 between the distal end 484 of the sleeve 480 and the necked portion 486 can function as a housing having an interior cavity for accommodating the spring clip 160. The housing can be sized and shaped to accept the spring clip 160 therein and allow the resilient arms 164 of the spring clip 160 to move between the ready to use position shown in FIG. 4A, and the protective position shown in FIG. 4C.

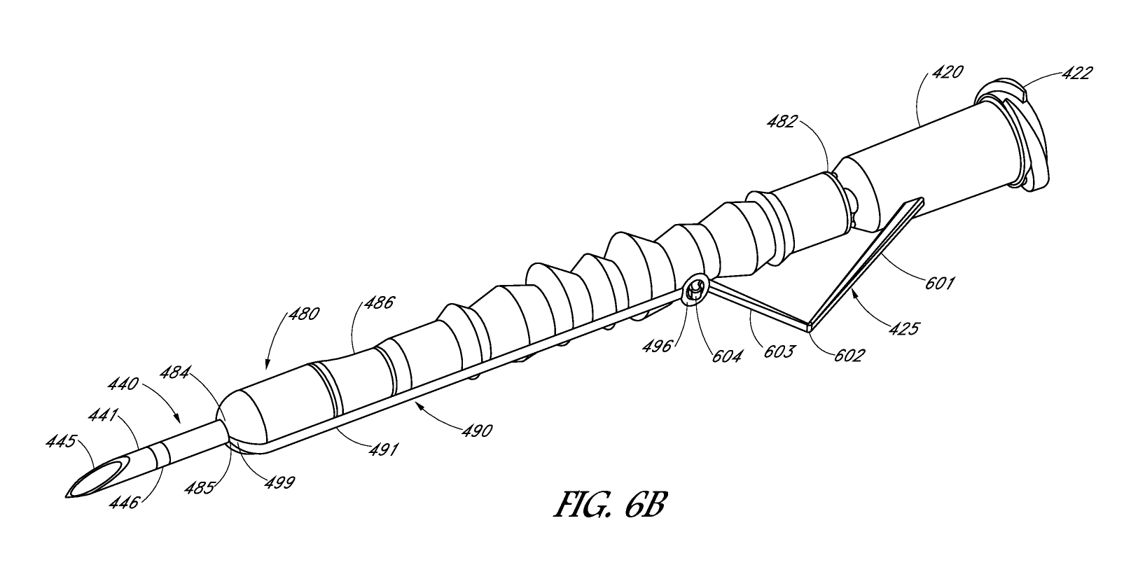

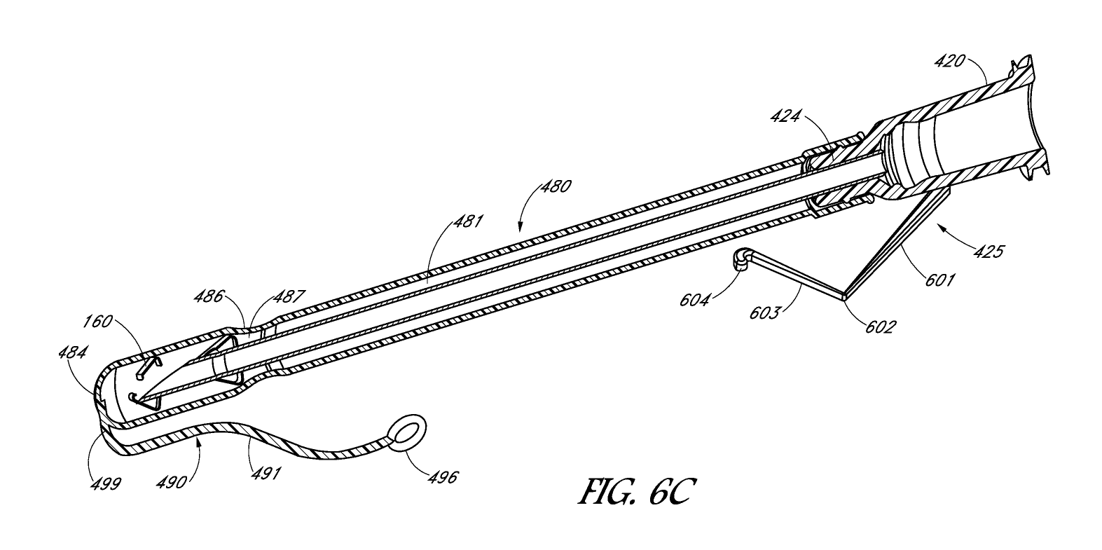

FIGS. 5A and 5B illustrate another embodiment of a safety needle assembly 500 provided in accordance with further aspects of the present disclosure. FIG. 5A illustrates an initial state of the needle assembly 500 in the ready to use position in which the needle tip 445 of the needle 440 is exposed. FIG. 5B shows the needle assembly in the protective position, such as after use and the safety needle assembly is activated. The present safety needle assembly 500 is similar to the safety needle assembly 400 of FIGS. 4A-4F with a few exceptions. In the present embodiment, the safety needle assembly 500 comprises a pair of securing devices 490 extending from opposite sides of the sleeve 480 and a pair of catches 425 extending from opposite sides of the needle adapter 420 engaging the pair of securing devices 490 to retain the elastic sleeve 480 under compression in the ready to use position. In the present embodiment, the pair of catches 425 can function as release buttons, which when activated by a user disengage the pair of catches 425 from the pair of securing devices 490. In one example, the pair of catches 425 can be activated by squeezing the catches 425 simultaneously towards each other. Thus, the safety needle assembly 500 of the present embodiment can be activated manually by a user to release the sleeve 480 and the spring clip 160 inside the sleeve to cover the needle tip 445 by activating the catches 425 to uncouple the securing devices 490.

Referring specifically to FIG. 5A, the safety needle assembly 500 comprises a needle adapter 420, a needle 440, a needle guard, spring clip or tip protector 160 slidably carried on the needle 440, a biasing member 480 in the form of an elastic sleeve 480 extending distally of the needle adapter 420 and enclosing the spring clip 160 and a portion of the needle 440 proximal of the needle tip, and the pair of securing devices 490 engaging a pair of corresponding catches 425 to compress at least a portion of the elastic sleeve 480 in a ready to use position. In an example, the entire length of the elastic sleeve 480 distal the needle adapter 420 is compressed.

The pair of catches 425 can each extend from opposite ends of the outer surface of the needle adapter 420 between the proximal end 422 and the distal end 424. In other examples, the pair of catches can extend from one of the ends. The overall shape of the needle adapter 420 may vary from the shape shown. As shown, at least some portions of an outer surface of the needle adapter 420 tapers inward from the proximal end 422 to the distal end 424 to form a partially frustoconical shaped contour that a user can readily handle or grip to attach the safety needle assembly 500 to a syringe or other medical devices. In other examples, a cross-section of the needle adapter 420 can be circular, rectangular, or any regular or irregular shape to form the grip. Interiorly, the needle adapter is sized as a female Luer to receive a male Luer tip. Surface features, such as fins and bumps, may be added to form a more effective gripping surface. The proximal end 482 of the sleeve 480 can be attached around a distal portion of the outer surface of the needle adapter 420. In other examples, adjacent the distal end 424 of the needle adapter 420 can be a ring shaped planar surface defining the ledge 424A for supporting the elastic sleeve 480. The shape and size of the needle adapter 420 is not limited.

The needle 440 comprises a needle shaft 441 having a proximal end attached to the needle adapter 420, a needle tip 445 formed at a distal end of the needle shaft 441, and, optionally, a change in profile 446 positioned adjacent the needle tip 445 on the needle shaft 441. The proximal end of the needle shaft 441 can be press fitted into the distal end 424 of the needle adapter 420 or glued into a glue well at the distal end 424 of the needle adapter 420. The needle tip 445 is configured to puncture the skin and tissue of a patient so that fluid can be injected into the patient, such as from a syringe attached to the needle adapter 420, or blood or other bodily fluid can be withdrawn through the needle tip 445 into the syringe barrel. The needle tip 445 can be cut or ground to have any known prior art tip geometries, including a non-coring needle tip. The optional change in profile 446 can have a cross-section larger than a cross-section of the needle shaft 441 at a nominal diameter of the needle shaft to prevent the spring clip 160 from detaching from the safety needle assembly 400, as will be discussed below in reference to FIG. 5B. The change in profile 446 can be a crimp, a bulge, a sleeve with a bore mounted over and secured to the needle, an attachment, or a material buildup.

The elastic sleeve 480 is provided with the needle 440 to move the spring clip or tip protector 160 in a distal direction to protect the needle tip 445 from unintended needle sticks following use. In the illustrated embodiment, the elastic sleeve 480 is a hollow tubular structure defining an interior cavity 481 having an opening 483 at a proximal end 482 and a partially enclosed distal end 484 having an aperture 485 for the needle 440 to extend therethrough in the ready to use position. The sleeve 480 can surround the needle 440 and the spring clip 160 with the needle tip 445 extending through the opening at the distal end of the sleeve in the ready to use position. To prevent the elastic sleeve 480 from separating from the needle adapter 420, the proximal end 482 of the elastic sleeve 480 can be secured to the needle adapter 420, such as by mechanical inter-engagement, glue, bonding, welding, ultrasonic welding, or other securement means. In some examples, the proximal end 482 of the sleeve 480 can be attached directly to the needle shaft 441 at or near the interface of the needle 440 and the needle adapter 420.

A necked portion 486 in the form of an internal shoulder adjacent the distal end 484 of the sleeve 480 and proximal of the spring clip 160 is configured to press against the spring clip 160, or more specifically, to a proximally facing surface 462A of a proximal wall 462 of the spring clip 160 to propel the spring clip 160 distally towards the needle tip 445 to shield the needle tip from inadvertent needle sticks, as further discussed below in reference to FIG. 5B. The internal shoulder can extend radially inwardly from a surface of the interior cavity 481 of the sleeve 480 towards the needle 440 and having a passage 487 or opening large enough for the needle shaft 441 to pass through but small enough to advance the spring clip 160 along the needle shaft 441. Thus, the size or diameter of the passage 487, which can resemble an orifice, can be slightly larger than the needle shaft 441 but less than the proximal wall 162 of the spring clip 160.

In one example, the necked portion 486 of the sleeve 480 presses against or abuts the spring clip 160 without mechanical inter-engagement so that the spring clip 160 can remain rotatable about the needle shaft 441 relative to the sleeve 480. In another example, the necked portion 486 is attached to the proximally facing surface 162A of the proximal wall 162 so that the spring clip 160 is not free to rotate about the needle shaft 141 relative to the sleeve 480. As shown in the figures, the sleeve 480 extends along nearly the entire length of the needle shaft 441 from the ledge 424a of the needle adapter 420.

Characteristics of the sleeve 480, such as the sleeve material and elastic properties of the sleeve 480, can be selected depending on the particular application of the safety needle assembly 500. For example, when a force is applied to the sleeve 480 to compress the sleeve 480 some distance, elastic energy is stored in the sleeve 480. When the force is removed, the sleeve 480 will elastically return to its original shape, similar to the sleeve 480 as discussed above for the safety needle assembly 400 of FIGS. 4A-4C.

The securing devices 490, which can be referred to as hold back straps, can each comprise an arm 491 extending from the outer surface of the sleeve 480 and an attach end 496 at a free end of the arm 491. The arm 491 and the attach end 496 can be integrally formed with the sleeve 480 or attached to the sleeve 480 by mechanical inter-engagement, glue, bonding, welding, or other securement means. The arm 491 of the securing device 490 extends proximally and may or may not extend radially outwardly with respect to the needle axis in its natural state from the sleeve 480. Moreover, the arm 491 may coil up in its natural state thereby shortening the overall length of the arm 491 when the securing device 490 disengages from a corresponding catch 425, as discussed below.

The arm 491 can extend outwardly from the outer surface of the sleeve 480 adjacent to or at the distal end 484 of the sleeve 480. However, the arm 491 can extend outwardly from the sleeve 480 adjacent to the necked portion 486 of the sleeve to prevent compression of the sleeve 480 surrounding the spring clip 160.

The catch 425 can be an elastic protrusion extending distal the proximal end 422 of the needle adapter 420 between the proximal end 422 and the distal end 424 of the needle adapter 420. The catch 425 can also extend at least partially radially outwardly in its natural state so that when the catch 425 is deflected inwardly towards the needle axis in an engaging position to hold the attach end 496 of the securing device 490 in the ready to use position, elastic potential energy can be stored in the catch 425.

A free end 426 of the catch 425 can directly contact a holding device 428 located on the needle adapter 420 or elastic sleeve 480 to maintain the catch 425 in the engaging position. The holding device 428 can be, for example, a notch on the needle adapter 420 or the sleeve 480, or the corner formed between the proximal end 482 of the sleeve 480 and the needle adapter 420. The catch 425 can spring from the engaging position to an activated position, which can be a position of the catch 425 in its natural state, by removing contact between the free end 426 of the catch 425 and the holding device 428, such as by squeezing the catches 425 towards each other. The catch 425 may also be a clamp or other holding device on the needle adapter 420 capable of maintaining the sleeve 480 under compression in the ready to use position by securing the attach end 496 of the securing device 490 and releasing the attach end of the securing device 490 to allow the sleeve 480 to expand to its normal state.

When the attach end 496 of the securing device 490 engages the catch 425, the elastic sleeve 480 is at least partially compressed to provide tension along the securing device 490 to maintain the attach end 496 to the catch 425, as further discussed below. More specifically, when the securing device 490 engages the catch 425, the sleeve 480 can be divided between an uncompressed portion 488 distal of an anchor point 499, where the arm 491 extends from the sleeve 480, and a compressed portion 489 proximal of the anchor point 499. The uncompressed portion 488 of the sleeve 480 can be defined between the anchor point 499 and the distal end 484 of the sleeve 480. The anchor point 499 can be located at the distal end 484 of the sleeve 480, in which case there is no uncompressed portion 488. The anchor point 499 may instead be near the necked portion 486 of the sleeve 480 so that only the portion of the sleeve 480 proximal of the anchor point 499 is allowed to compress. The compressed portion 489 of the spring can be defined between the proximal end 482 of the sleeve 480 and the anchor point 499. As shown, nearly the entire length of the sleeve 480 proximal of the spring clip 160 is under compression. In another example, at least 80% of the entire uncompressed length of the sleeve 480 can be under compression or, put in another way, the uncompressed portion can be less than about 20% of the entire length of the sleeve 480. In yet another example, at least 50% of the entire length of the sleeve 480 can be under compression or, put in another way, the uncompressed portion of the spring can be less than about 50% of the entire length of the sleeve 480.

The arm 491 of the securing device 490 may normally be straight or coiled so that when the attach end 496 of the securing device 490 engages the catch 425, elastic potential energy is stored in the arm 491 of the securing device 290. That is, when the arm 491, whether normally straight or coiled up, is engaged with the catch 425 in the initial position, the arm 491 is simultaneously under tension caused by compressing the compressed portion 489 of the sleeve 480 thereby elastically stretching out the arm 491 to be more straight or less coiled or bent. Elastic potential energy can be stored in the arm 491 from the elastic deflection. The tension in the arm 491 caused by the sleeve 480 under compression can maintain the engagement between the attach end 496 and the catch 425 and prevents the arm 491 and the attach end 496 from elastically returning outwardly away from the sleeve 480. Thus, the sleeve 480 can be maintained under compression in the ready to use position by the securing device 490, which functions as a hold back strap.

In some examples, the attach end 496 can comprise a loop, a hook, a lever, a lip, a notch, a shoulder, a spline, or combinations thereof. The attach end 496 can removably engage the catch 425 and upon compression of the sleeve 480, separate and pull away from the catch 425 by elongation of the sleeve 480 as it returns to its normal state, as further discussed below. The removable engagement can be a simple overlap with friction, a positive engagement with part of the catch 425 entering part of the attach end 496, or combinations thereof. Because the compressed portion 489 of the sleeve 480 is held in a compressed state by the engagement of the holding device 428 with the catch 425, the safety needle assembly 500 is in an equilibrium state in the ready to use position of FIG. 5A.

With reference to FIG. 5B, when the catches 425 are activated, the catches 425 break contact with the holding device 428 thereby releasing the attach end 496 of the securing device 490. Depending on the structure of the catch 425, the catch 425 can elastically spring outwardly away from the securing device 490 due to the stored elastic potential energy and thereafter remain out of the reach of the securing device 490. Once the attach end 496 is no longer held by the catch 425, the sleeve 480 can be restored to its natural state and push the spring clip 160 inside the sleeve 480 to move distally to cover the needle tip 445.

The sleeve confines the spring clip 160 and prevents the spring clip 160 from moving distally in the ready to use position. The spring clip 160 is configured to slide along the needle shaft 441 to shield the needle tip 445 from unintended needle sticks following activation, such as following use and the attach end 496 is separated from the catch 425, as discussed above for FIGS. 1A-1C. The spring clip 160 can slide proximally from the initial or ready to use position shown in FIG. 5A to the after-use, closed, protective, or secured position shown in FIG. 5B where the spring clip 160 is effectively located over the needle tip 445 to shield the needle tip 445, such as after the needle 440 is removed from the patient.

As shown, one embodiment of the spring clip 160 of the present disclosure comprises a proximal wall 162 and two resilient arms 164 extending distally from the proximal wall 162, as discussed above for FIGS. 1A-1C. As shown, the tips 168 of the two resilient arms 164 press against the needle shaft 441 in the ready to use position of FIG. 1A and the in-use position shown in FIG. 5B. The resilient arms 164 are elastically deflected outwardly by the needle shaft 141 and the ends 166 of the distal walls 165 are pressed against diametrically opposite points of the needle shaft 141.

With further reference to FIG. 5B, as the needle is 440 is removed from the patient, the spring clip 160 is urged distally along the needle shaft 441 by the necked portion 486 of the sleeve 480 pressing against the proximal wall 162 of the spring clip 160 until the proximal wall 162, and more specifically the perimeter defining the opening 163 on the proximal wall 162, contacts the change in profile 446 on the needle 440, which stops further distal movement of the spring clip 160 and further expansion of the sleeve 480. In other examples, the length of the sleeve is sized and shaped to provide the constraint against further distal movement of the spring clip. Before, concurrently, or shortly thereafter, the resilient arms 164 of the spring clip 160, now no longer pressing against the needle shaft 441, activate and move radially to move the two distal walls over the needle tip 445 to cover the needle tip 445 in the secured position. In the secured position, the free ends 166 or the tips 168 of the resilient arms 164 slide off the needle shaft 441 and elastically move towards each other to cover the needle tip 445 to prevent inadvertent needle sticks. In some examples, the distal walls can each have folded tabs, such as two or more folded tabs, to define a holding space for capturing the needle tip therein to prevent the needle tip from moving laterally outside the side edges of the resilient arms 164. The resilient arms 164 can also have different lengths so that when the spring clip 160 is activated, the two distal walls 165 are axially offset and covering the needle tip 445. If no change in profile 446 on the needle 440 is provided, the distal end 484 of the sleeve 480 stops further distal movement of the spring clip 160 when the spring clip 160 is activated and covering the needle tip 445.

The engagement between the change in profile 446 and the opening 163 in the proximal wall 162 of the spring clip 160 or the distal end 484 of the sleeve prevents the spring clip 160 from displacing distally off of the needle 440. Specifically, the distally facing surface 162B, such as the perimeter defining the opening 163, of the proximal wall 162 abuts against the change in profile 446 to prevent further distal movement of the spring clip 160 following movement to the used or protected position in which the needle tip is shielded. Because the change in profile 446 is larger in size than the opening 163, the spring clip 160 cannot slide past the change in profile, thereby confining the needle tip 445 within the spring clip 160 and preventing unintended needle sticks after the needle 440 has been removed from the patient. Similarly, because the aperture 485 at the distal end 484 of the sleeve 480 is smaller than the spring clip 160, the spring clip 160 is confined within the interior cavity 481 of the sleeve 480 between the distal end 484 of the sleeve 480 and the necked portion 486. The sleeve 480 can also prevent or significantly reduce the likelihood of blood exposure when covering the needle tip 445. The distal portion of the sleeve 480 between the distal end 484 of the sleeve 480 and the necked portion 486 can function as a housing having an interior cavity for accommodating the spring clip 160. The housing can be sized and shaped to accept the spring clip 160 therein and allow the resilient arms of the spring clip 160 to move between the ready to use position shown in FIG. 5A, and the protective position shown in FIG. 5B.

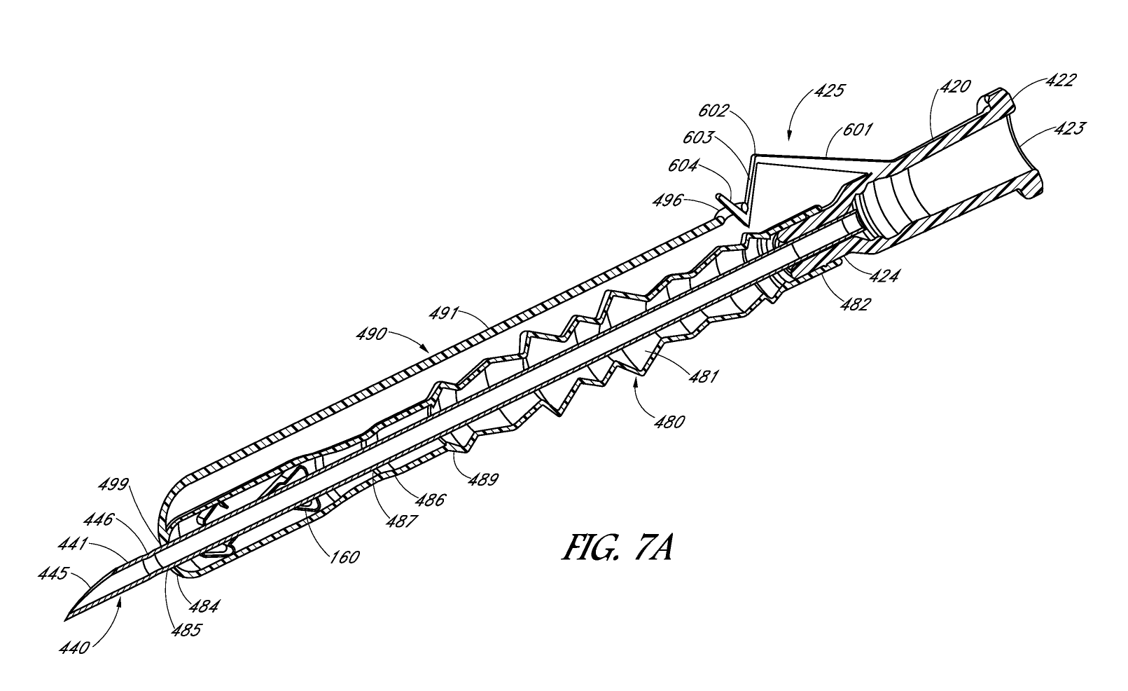

FIGS. 6A-6C illustrate another embodiment of a safety needle assembly 600 provided in accordance with further aspects of the present disclosure. FIG. 6A illustrates an initial state of the needle assembly 600 in the ready to use position in which the needle tip 445 of the needle 440 is exposed, FIG. 6B shows the needle assembly 600 during use, and FIG. 6C shows a cross-sectional view of the needle assembly 600 after use in the protective position. The present safety needle assembly 600 is similar to the safety needle assembly 500 of FIGS. 5A and 5B in that a catch 425 extends from a side of the needle adapter 420, engages a securing device 490, and functions as a release button, which when activated by a user, disengages the catch 425 from the securing device 490 to release the sleeve 480. Thus, the safety needle assembly 600 can be activated when a user intervenes to release the sleeve 480 thereby allowing the sleeve 480 and the spring clip 160 inside the sleeve 480 to cover the needle tip 445 in the protective position upon needle 440 removal, such as by activating the catch 425. However, unlike the safety needle assembly 500 of FIGS. 5A and 5B, the sleeve 480 of the present safety needle assembly 600 can move along the needle shaft 441 as the needle 440 is inserted into the patient. Thus, the present safety needle assembly 600 shares some aspects of the safety needle assembly 400 of FIGS. 4A-4F, which features a sleeve 480 having a distal end 484 that is movable to further compress the sleeve 480.

Referring to FIG. 6A, in the present safety needle assembly 600, the catch 425 extends from the needle adapter 420 and forms a generally L-shaped structure comprising a first segment 601 extending from the needle adapter 420, a second segment 603, a flexible elbow 602 coupling the second segment 603 to the first segment 601, and a coupler 604 at a free end of the second segment 603 engaging the attach end 496 of the securing device 490. The shape of the coupler 604 and the attach end 496 is not limited. For example, the coupler 604 can be a tab, a hook, a protrusion, or spline for engaging the attach end 496, which can be a complementary hook, lever, lip, notch, shoulder, spline, or combinations thereof. As shown, the attach end can be ring-shaped and the coupler 604 can form a curved resilient hook with a lip of the curved hook extending outwardly, such that the hook-shaped coupler can be squeezed inside a bore of the attach end 496 to form an interference fit when engaged in the ready to use position.

With reference to FIG. 6B, when the needle 440 is inserted into the patient, the distal end 484 of the sleeve 480 pushes against the skin of the patient to move the distal end 484 of the sleeve 480, the spring clip 160, and the securing device 490 proximally toward the catch 425, thereby further compressing the sleeve 480 from its initial compressed state in the ready to use position, as discussed above for the needle assembly 400 of FIGS. 4A-4F. The securing device 490 may be rigid and hence move the coupler 604 as the securing device 490 advances proximally on the needle 440, thereby causing the elbow 602 to bend as the angle between the first segment 601 and the second segment 603 decreases. At this moment, the coupler 604 can still maintain contact with the attach end 496 until the catch 425 is activated by the user as shown in FIG. 6C. The flexible elbow 602 may be elastic and therefore capable of storing elastic energy as the elbow 602 bends from the moving the second segment 603 towards the first segment 601. Said differently, as the angle formed between the first and second segments 601, 603 decreases, the potential elastic energy stored in the elbow increases. Thus, the elbow 602 can provide a resistive force against the securing device 490 to maintain a positive engagement between the coupler 604 and the attach end 496 as the securing device 490 moves proximally towards the catch 425.

Alternatively, the securing device 490 may instead be flexible and not rigid. In this example, as the distal end 484 of the sleeve 480 moves proximally while the sleeve 480 compresses, because the securing device 490 is flexible and not rigid, the catch 425 does not flex at the elbow during sleeve compression. Instead, tension is maintained in the securing device 490 between the sleeve 480 and the catch 425 in the ready to use position and during use to maintain engagement between the coupler 604 of the catch 425 and the attach end 496 of the securing device 490. The distal end 484 of the sleeve 480 and consequently the spring clip 160 inside the distal portion of the sleeve 480 is free to slide along the needle shaft 441 as the needle is inserted into the patient and removed from the patient while maintaining tension in the securing device 490. Once the needle assembly 600 is removed from the patient, the user can activate the catch 425 to release the attach end 496 from the coupler 604 so that the sleeve 480 can extend to move the spring clip 160 to cover the needle tip 445. The user can activate the catch 425 by pressing on the first segment 601, the elbow 602, or the second segment 603, or combinations thereof. Alternatively, the catch 425 can be activated by pressing the elbow or other parts of the catch against a surface, such as a table top.

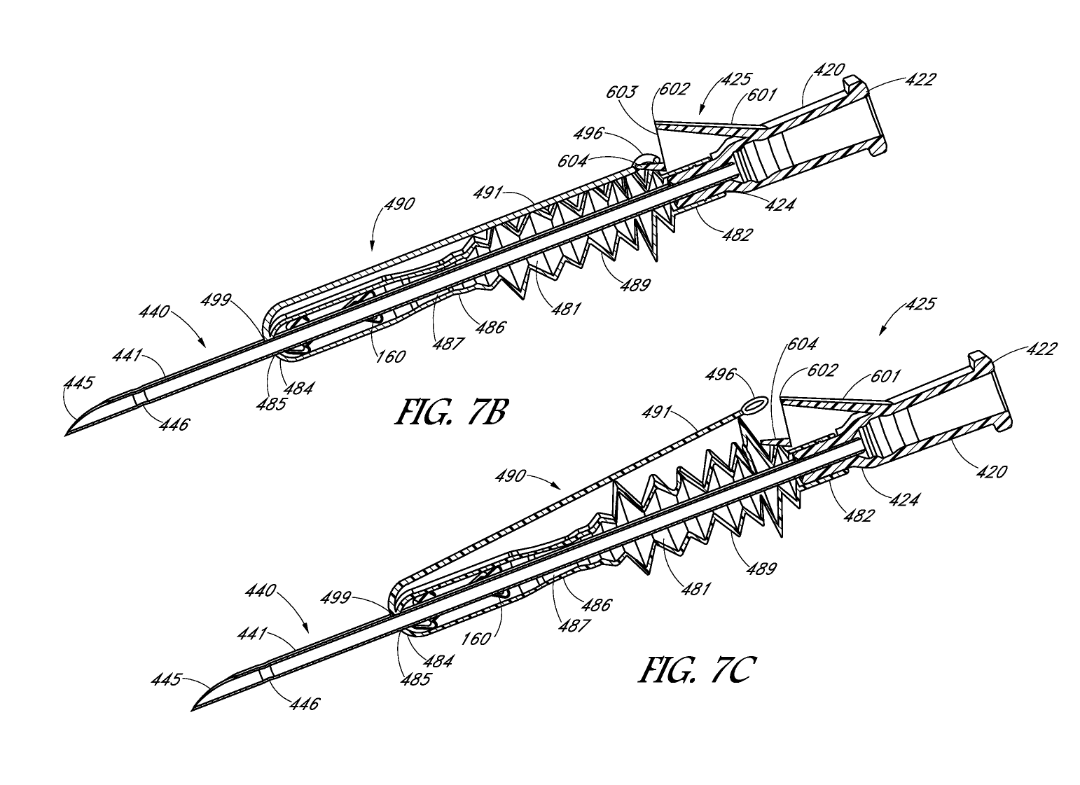

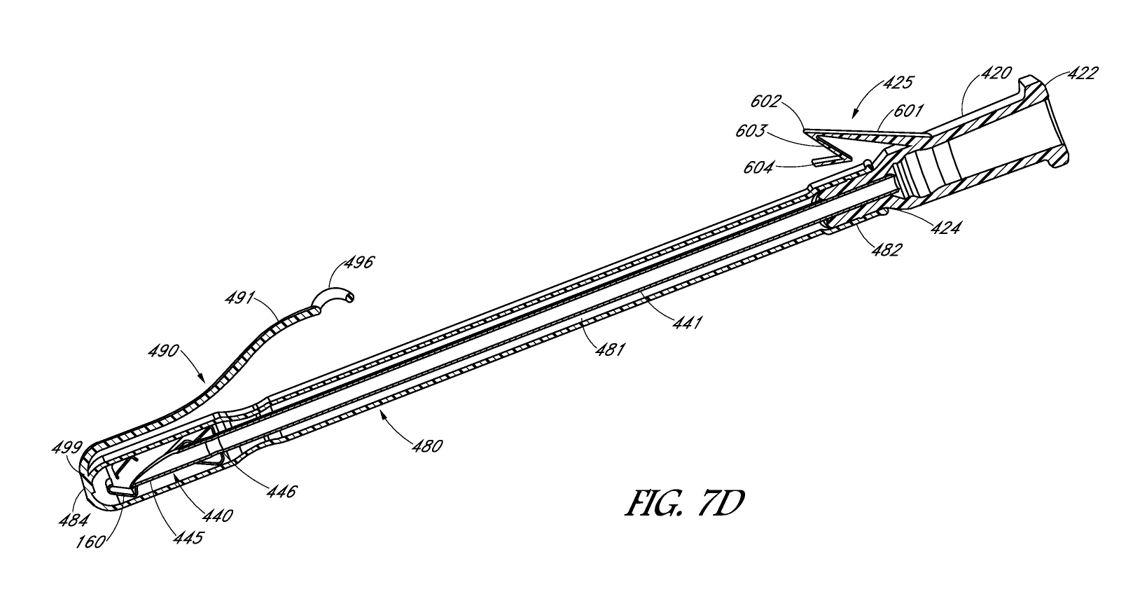

FIGS. 7A-7D illustrate another embodiment of a safety needle assembly 700 provided in accordance with further aspects of the present disclosure. FIG. 7A shows a cross-sectional view of the needle assembly 700 in the ready to use position in which the needle tip 445 of the needle 440 is exposed. FIG. 7B shows the sleeve being compressed during use and FIG. 7C shows a segment or section of the sleeve expanding radially as the sleeve is further compressed to disengage the catch from the securing device of the needle assembly 700. FIG. 7D shows a cross-sectional view of the needle assembly 700 after use and the needle and/or sleeve in the protective position. The sleeve 480 has biasing properties and can be considered a biasing element or biasing member.

The present safety needle assembly 700 is similar to the safety needle assembly 600 of FIGS. 6A-6C except that the catch 425 extending from a side of the needle adapter 420 can optionally be activated automatically without a separate activation step. In an example, changes in the shape of the sleeve during proximal movement of the sleeve 480 as the needle 440 is inserted into the patient can be used to activate the catch. Similar to the embodiment of FIGS. 4A-4C, the safety needle assembly 700 of the present embodiment can be activated during insertion of the needle 440 into the patient, thereby allowing the sleeve 480 and the spring clip 160 inside the sleeve 480 to cover the needle tip 445 in the protective position during needle 440 removal. The sleeve 480 of the present safety needle assembly 700 can move along the needle shaft 441 as the needle 440 is inserted into the patient, such as when pushed by the skin during needle insertion. Thus, the present safety needle assembly 700 shares some aspects of the safety needle assembly 400 of FIGS. 4A-4C, which features a sleeve 480 having a distal end 484 that is movable to further compress the sleeve 480.

Referring to FIG. 7A, in the present safety needle assembly 700, the catch 425 extends from the needle adapter 420 and forms a generally L-shaped structure comprising a first segment 601 extending from the needle adapter 420, a second segment 603, a flexible elbow 602 coupling the second segment 603 to the first segment 601, and a coupler 604 at a free end of the second segment 603 engaging the attach end 496 of the securing device 490. The shape of the coupler 604 and the attach end 496 is not limited. For example, the coupler 604 can be a tab, a hook, a protrusion, or spline for engaging the attach end 496, which can be a complementary hook, lever, lip, notch, shoulder, spline, or combinations thereof. As shown, the attach end 496 can be shaped as a ring or an open hook, and the coupler 604 can form an L-shaped hook with the second segment 603, such that the coupler 604 can extend into a bore of the attach end 496 to maintain engagement with the attach end 496 in the ready to use position.

With reference to FIG. 7B, when the needle 440 is inserted into the patient, the distal end 484 of the sleeve 480 pushes against the skin of the patient to move the distal end 484 of the sleeve 480, the spring clip 160, and the securing device 490 proximally toward the catch 425, thereby further compressing the sleeve 480 from its initial compressed state in the ready to use position, as discussed above for the needle assembly 400 of FIGS. 4A-4F. The securing device 490 may be sufficiently rigid to move the coupler 604 by pushing against the second segment 603 as the securing device 490 advances proximally on the needle 440, thereby causing the elbow 602 to bend as the angle between the first segment 601 and the second segment 603 decreases. Simultaneously, as the angle between the first segment 601 and the second segment 603 decreases, the coupler 604 may move away from out of the bore of the attach end 496 until the coupler 604 is fully disengaged from the attach end 496.

Referring now to FIG. 7C, as the sleeve 480 compresses further from its initial compressed state in the ready to use position, a bulge or enlarged region or section grows radially outward relative to the length of the sleeve 480. Said differently, as the needle 440 extends deeper into the patient, the outer dimension or diameter of the sleeve 480, or at least parts or sections of the sleeve, increases as the distal end 484 of the sleeve 480 moves proximally to compress the sleeve 480. As the outer dimension of the sleeve 480 grows radially, the sleeve 480 eventually pushes against the arm 491 of the securing device 490 until the attach end 496 at the end of the arm 491 is moved away from the coupler 604 of the catch 425, thereby disengaging the coupler 604 from the catch 425.

Referring to FIG. 7D, as the needle is retracted from the patient, the distal end 484 of the sleeve 480 presses against the skin of the patient until the needle 440 is fully removed with the sleeve 480 and the spring clip 160 covering the needle tip 445 in the protected position.

Methods of making and of using the needle assemblies and their components described elsewhere herein are contemplated and are considered within the scope of the present disclosure.

The above description presents various embodiments of the present invention, and the manner and process of making and using them, in such full, clear, concise, and exact terms as to enable any person skilled in the art to which it pertains to make and use this invention. This invention is, however, susceptible to modifications and alternate constructions from that discussed above that are fully equivalent. Consequently, this invention is not limited to the particular embodiments disclosed. On the contrary, this invention covers all modifications and alternate constructions coming within the spirit and scope of the invention as generally expressed by the following claims, which particularly point out and distinctly claim the subject matter of the invention.

1. A safety needle assembly, comprising:
a needle adapter having an opening at a proximal end and a distal end;
a needle extending distally from the distal end of the needle adapter, the needle having a needle shaft and a needle tip at a distal end of the needle shaft;
a spring clip slidably riding on the needle shaft and having a proximal wall with an opening for the needle shaft to pass therethrough, the spring clip urging against the needle shaft adjacent the needle tip in a ready to use position, and shielding the needle tip in a secured position;
a biasing member having a proximal end coupled to the needle adapter and a distal end coupled to the proximal wall of the spring clip; and
a securing device under tension and coupled between the biasing member and the needle adapter in the ready to use position, and decoupled when the spring clip moves in a proximal direction.

2. The safety needle assembly of claim 1, wherein the needle further comprises a change in profile adjacent the needle tip on the needle located distal of the proximal wall of the spring clip, the opening of the spring clip has a size smaller than a size of the change in profile to prevent removing the spring clip from the needle, and the distal end of the biasing member is adjacent the change in profile in the secured position.

3. The safety needle assembly of claim 2, wherein the spring clip is urging against opposite sides of the needle shaft when not in the secured position.

4. The safety needle assembly of claim 2, wherein the spring clip further comprises a pair of resilient arms extending distally from the proximal wall, the resilient arms urging against the opposite sides of the needle shaft, and the resilient arms shielding the needle tip in the secured position.

5. The safety needle assembly of claim 1, wherein the securing device comprises an attach end coupled to a latch part of the biasing member to secure the spring clip in the ready to use position, wherein the attach end elastically moves away from the biasing member when the attach end is decoupled from the biasing member.

6. The safety needle assembly of claim 5, wherein the attach end is decoupled from the biasing member when the latch part moves proximally away from the attach end.

7. The safety needle assembly of claim 5, wherein the attach end is decoupled from the biasing member when a decoupler of the biasing member urges proximally against the attach end.

8. The safety needle assembly of claim 5, wherein the latch part of the biasing member is a coil and the decoupler of the biasing member is an adjacent coil.

9. The safety needle assembly of claim 5, wherein the securing device comprises more than one attach end.

10. The safety needle assembly of claim 1, wherein the biasing member extends nearly an entire length of the needle shaft in the ready to use position.

11. A method of making a safety needle assembly, comprising:
extending a needle distally from a distal end of a needle adapter through a biasing member and an opening in a proximal wall of a spring clip, the needle adapter having an opening at a proximal end, the needle having a needle shaft and a needle tip at a distal end of the needle shaft;
urging the spring clip against the needle shaft adjacent the needle tip in the ready to use position;
coupling a proximal end of the biasing member to the needle adapter;
contacting a distal end of the biasing member with the proximal wall of the spring clip; and
compressing a portion of the biasing member with a securing device coupled to a part of the biasing member in the ready to use position, the securing device decouples to release the spring clip to shield the needle tip in a secured position, and wherein the securing device decouples by proximal movement of the spring clip.

12. The method of claim 11, wherein the needle further comprises a change in profile adjacent the needle tip on the needle located distal of the proximal wall of the spring clip, the opening of the spring clip has a size smaller than a size of the change in profile to prevent removing the spring clip from the needle, and the distal end of the biasing member is adjacent the change in profile in the secured position.

13. The method of claim 12, wherein the spring clip is urging against opposite sides of the needle shaft when not in the secured position.

14. The method of claim 13, wherein the spring clip further comprises a pair of resilient arms extending distally from the proximal wall, the resilient arms urging against the opposite sides of the needle shaft, and the resilient arms shielding the needle tip in the secured position.

15. The method of claim 11, wherein the securing device comprises an attach end coupled to the latch part of the biasing member to secure the spring clip in the ready to use position, wherein the attach end elastically moves away from the biasing member when the attach end is decoupled from the biasing member.
